(19) United States
(12) Patent Application Publication
(10) Pub. No.: US 2026/0263624 A1
(43) Pub. Date: Sep. 10, 2026

(54) COMBINATION OF ANTIBODY-DRUG CONJUGATE AND CANCER THERAPY, AND USE THEREOF

(71) Applicants: GENEQUANTUM HEALTHCARE (SUZHOU) CO., LTD., Suzhou (CN); GENEQUANTUM MEDICINE (SUZHOU) CO., LTD., Suzhou (CN)

(72) Inventors: Gang QIN, Suzhou (CN); Paul H. SONG, Suzhou (CN); LiLi SHI, Suzhou (CN)

(21) Appl. No.: 19/167,314

(22) PCT Filed: Mar. 20, 2024
(86) PCT No.: PCT/CN2024/082634

§ 371 Date: Sep. 19, 2025

(30) Foreign Application Priority Data
Mar. 21, 2023 (WO) PCT/CN2023/082677

(51) Int. Cl. A61K 47/68; A61K 31/337; A61K 31/4709; A61K 31/517; A61K 31/7068; A61P 35/00; A61P 35/04

(52) U.S. Cl. CPC A61K 47/6855; A61K 31/337; A61K 31/4709; A61K 31/517; A61K 31/7068; A61K 47/68033; A61P 35/00; A61P 35/04

(57) ABSTRACT

Provided are a combination of antibody-drug conjugate and cancer therapy (especially kinase inhibitor or chemotherapy), and use thereof.

### TECHNICAL FIELD

The present disclosure relates to the biopharmaceutical field, in particular, a combination of antibody-drug conjugate and cancer therapy (especially kinase inhibitor or chemotherapy), and use thereof.

### BACKGROUND

Human epidermal growth factor receptor-2 (HER2) belongs to the epidermal growth factor (EGFR) receptor tyrosine kinase family. In addition to the activation of molecular pathways responsible for cell proliferation and survival under stress conditions, HER2 directly regulates programmed cell death. HER2 overexpression or amplification characterizes a number of common cancers. HER2-targeted cancer therapies in the market include antibodies, tyrosine kinase inhibitors and antibody-drug conjugates (ADCs).

HER2-targeted ADCs have provided a promising new approach to the treatment of various cancers including breast cancer, gastric or gastroesophageal junction cancer, colorectal cancer, non-small cell lung cancer and other HER2-positive cancers. So far, anti-microtubules agents such as tubulin polymerization inhibitors (e.g., maytansinoids and auristatins) represent the large majority of payloads developed. For example, DM1, which is a maytansinoid, is used as the payload in T-DM1 (Ado-trastuzumab emtansine, Kadcyla®, Genentech/Roche) and in several investigatory ADCs.

However, on one hand, T-DM1, as well as the other commercially available ADCs and most of the ADCs in clinical trials, are synthesized by chemical conjugation, using a thiosuccinimide structure (thiosuccinimide linkage) to conjugate the small molecule drug with the targeting antibody or protein. The thiosuccinimide structure is formed by the reaction of a thiol group and a maleimide. The thiosuccinimide linkage is not stable. In organisms, reverse Michael addition or exchange with other thiol groups leads to the fall-off of the payload from the ADC and off-target toxicity, which reduces the safety. On another hand, chemical coupling reaction is not site specific, and the resulting ADCs show high heterogeneity. We have a long interest in the development of stable linkers, which bears the fruits including a prolonged half life of ADC drugs in systemic circulation, decreased premature fall-off and increased accumulation of the payload in the target. Our HER-2 targeting ADCs, including GQ1001 (see CN106856656B), have demonstrated improved safety profiles.

Drug resistance is another major hurdle in the development for cancer therapies. Intrinsic resistance and acquisition of new resistance mutations are observed with conventional chemotherapy, targeted and immunological therapies. Compensatory adaptation processes in the oncogenic pathway(s) is also described as a resistance mechanism. Intensive research is done to overcome this hurdle. Development of combined therapies are frequently confronted with limited clinical efficacy, more complicated regimens, pharmacological compatibility issues and therefore requirement for a higher level of risk management. Cost-effectiveness is also a factor to consider.

For the treatment of HER2-positive breast cancer, dual HER2 blockade using trastuzumab and pertuzumab, combined with chemotherapy (such as taxanes and platinum agents) is a standard first-line treatment under several circumstances, but development of resistance to trastuzumab is inevitable. Combinations comprising the tyrosine kinase inhibitor tucatinib, neratinib or lapatinib are used mainly in second-line or third- or later-line treatments. The tyrosine kinase inhibitor pyrotinib is approved in 2018 for use in the treatment (in combination with capecitabine) of HER2-positive, advanced or metastatic breast cancer in patients previously treated with anthracycline or taxane chemotherapy, and approved in 2022 for use in neoadjuvant treatment (in combination with trastuzumab plus docetaxel) of HER2-positive early stage or locally advanced breast cancer (LABC). T-DM1 as monotherapy can be used for patients with metastatic breast cancer (mBC) who have received one or more chemotherapy agents, and can be used as an adjuvant treatment for patients of breast cancer with HER2 overexpression who have received anthracycline containing adjuvant chemotherapy and radiotherapy (if applicable). Although T-DM1 is widely studied as a skeleton drug for combination strategies in mBC theoretically, the side effects of therapies comprising T-DM1 limit its clinical investigation and application. For example, thrombocytopenia was observed in around 30-55% of patients who received single-agent T-DM1. The combination of T-DM1 and docetaxel was studied in patients with mBC or LABC, but nearly half of patients experienced dose reductions due to severe AEs (grade 3-4 neutropenia in 72% mBC). The combination of T-DM1 with pertuzumab showed only noninferior, but not superior, than taxane (docetaxel or paclitaxel) plus trastuzumab in the phase III MARIANNE study. Currently, clinical trials on combinations of T-DM1 with other agents (e.g., immune checkpoint inhibitors, CDK4/6 inhibitors and TKIs) are underway, but the side effect is still the main concern. The combined regimen comprising T-DM1 is associated with a higher incidence of side effects, and therefore the dose and number of treatment cycles are limited. Furthermore, resistance to chemotherapy drugs and/or the antibody affects the objective response rate. As an overall result, the benefits for the patient are limited.

In addition, T-DM1 only showed limited clinical activity in gastric or gastroesophageal junction cancer and failed to demonstrate superior efficacy compared with taxane (docetaxel or paclitaxel) in the phase II/III GATSBY study.

New therapies with high efficacy and less toxicity are still in urgent need.

### SUMMARY

In a first aspect, provided is a pharmaceutical combination, comprising the following (a) and (b);
(a) one or more conjugates having the structure of formula (i-1) and/or formula (i-2)

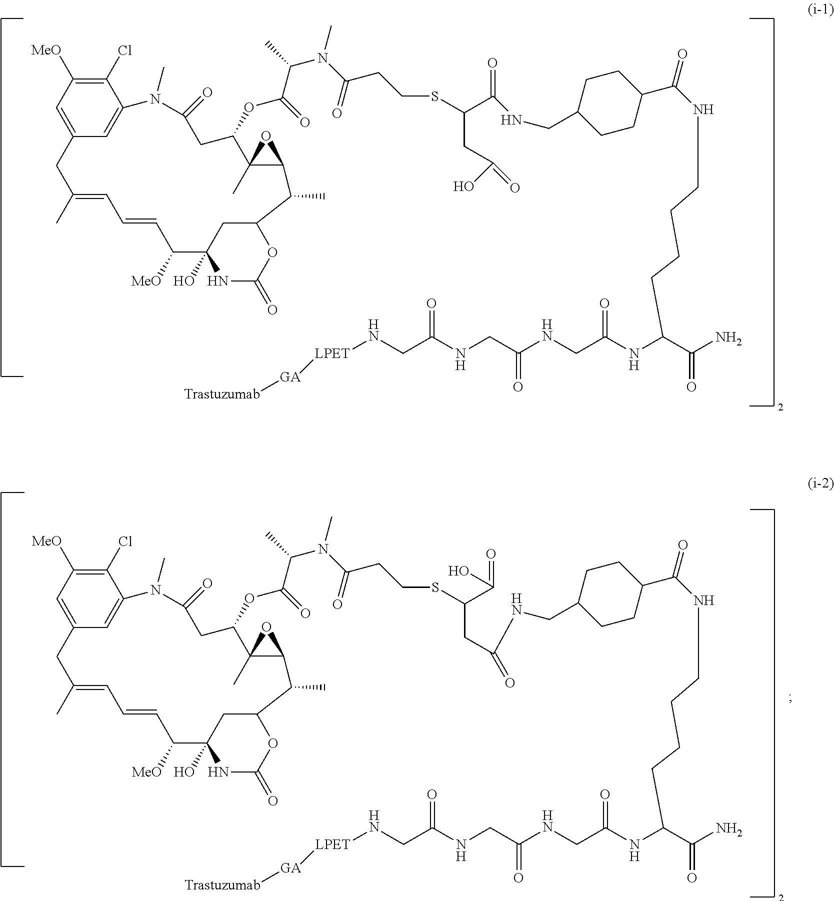

preferably, trastuzumab is connected to the rest moiety of the conjugate via the C-terminal of both of the light chains of trastuzumab;
(b) one or more additional cancer therapeutic agents selected from the group consisting of: tyrosine kinase inhibitors, chemotherapeutic agents, and pyrimidine antimetabolites.

In a second aspect, provided is a kit or a pharmaceutical composition, comprising the pharmaceutical combination of the present disclosure.

In a third aspect, provided is a method for preventing, alleviating or treating a cell proliferative disorder, comprising administering a subject in need of such treatment an effective amount of the pharmaceutical combination of any one of the present disclosure or the kit or pharmaceutical composition of the present disclosure.

### BRIEF DESCRIPTION OF THE DRAWINGS

FIG. 1a shows the efficacy of the combination of GQ01 with tucatinib in human breast cancer PDX model.

FIG. 1b shows the efficacy of the combination of GQ01 with neratinib in human breast cancer PDX model.

FIG. 1c shows the efficacy of the combination of GQ01 with pyrotinib in human breast cancer PDX model.

FIG. 2a shows the efficacy of the combination of GQ01 with tucatinib in human breast cancer PDX model.

FIG. 2b shows the efficacy of the combination of GQ01 with pyrotinib in human breast cancer PDX model.

FIG. 3a shows the efficacy of the combination of GQ01 with tucatinib in human breast cancer PDX model.

FIG. 3b shows the efficacy of the combination of GQ01 with pyrotinib in human breast cancer PDX model.

FIG. 4a shows the efficacy of the combination of GQ01 with tucatinib in human gastric cancer PDX model.

FIG. 4b shows the efficacy of the combination of GQ01 with pyrotinib in human gastric cancer PDX model.

FIG. 5a shows the efficacy of the combination of GQ01 with paclitaxel in human breast cancer CDX model.

FIG. 5b shows the efficacy of the combination of GQ01 with capecitabine in human breast cancer CDX model.

FIG. 6a shows the efficacy of the combination of GQ01 with paclitaxel in human breast cancer CDX model.

FIG. 6b shows the efficacy of the combination of GQ01 with capecitabine in human breast cancer CDX model.

### DETAILED DESCRIPTION

The specific embodiments are provided below to illustrate technical contents of the present disclosure. Those skilled in the art can easily understand other advantages and effects of the present disclosure through the contents disclosed in the specification. The present disclosure can also be implemented or applied through other different specific embodiments. Various modifications and variations can be made by those skilled in the art without departing from the spirit of the present disclosure.

### Definitions

Unless otherwise defined hereinafter, all technical and scientific terms used herein have the same meaning as commonly understood by those skilled in the art. The techniques used herein refer to those that are generally understood in the art, including the variants and equivalent substitutions that are obvious to those skilled in the art. While the following terms are believed to be readily comprehensible by those skilled in the art, the following definitions are set forth to better illustrate the present disclosure. When a trade name is present herein, it refers to the corresponding commodity or the active ingredient thereof. All patents, published patent applications and publications cited herein are hereby incorporated by reference.

The terms used herein to describe the outcome measures for the experimental drugs or pharmaceutical combinations can be referred to RECIST, v1.1 (Response Evaluation Criteria in Solid Tumours), or have the same meaning as commonly understood by those skilled in the art. In particular, response status can be describe using the following terms: CR, PR, PD and SD. Complete response (CR): disappearance of all target lesions; any pathological lymph nodes (whether target or non-target) must have reduction in short axis to <10 mm. Partial response (PR): at least a 30% decrease in the sum of diameters of target lesions, taking as reference the baseline sum diameters. Progressive disease (PD): at least a 20% increase in the sum of diameters of target lesions, taking as reference the smallest sum on study (this includes the baseline sum if that is the smallest on study); in addition to the relative increase of 20%, the sum must also demonstrate an absolute increase of at least 5 mm. The appearance of one or more new lesions is also considered progression. Stable disease (SD): neither sufficient shrinkage to qualify for PR nor sufficient increase to qualify for PD, taking as reference the smallest sum diameters while on study. In addition, terms “clinical complete response (cCR, complete response assessed using clinical methods)” and “pathological complete response (pCR or pathCR, complete response assessed using pathological methods)” can be used to describe the outcome in some embodiments.

The terms used herein to describe the diagnosis and treatment methods for humans can be referred to those latest Guidelines published by authority organizations, especially CSCO (Chinese Society of Clinical Oncology) or American Society of Clinical Oncology (ASCO), or have the same meaning as commonly understood by those skilled in the art.

Unless the context clearly dictates otherwise, singular forms like “a” and “the” include the plural forms. The expression “one or more” or “at least one” may mean 1, 2, 3, 4, 5, 6, 7, 8, 9 or more. When the term “therapy” is used in combination with other terms, such expressions are open-ended. For example, the term “palbociclib therapy” does not exclude use of other therapeutic agents. Other similar expressions such as “pyrotinib therapy” should also be understood in a similar manner.

The terms “about” and “approximately”, when used in connection with a numerical variable, generally mean that the value of the variable and all values of the variable are within experimental error (for example, within a 95% confidence interval for the mean) or within +10% of a specified value, or a wider range.

The term “optional” or “optionally” means the event described subsequent thereto may, but not necessarily happen, and the description includes the cases wherein said event or circumstance happens or does not happen.

The expressions “comprising”, “including”, “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprising” encompasses the expressions “consisting essentially of” and “consisting of”.

As used herein, the term “antibody” is used in a broad way and particularly includes intact monoclonal antibodies, polyclonal antibodies, monospecific antibodies, multispecific antibodies (e.g., bispecific antibodies), and antibody fragments, as long as they have the desired biological activity. The antibody may be of any subtype (such as IgG, IgE, IgM, IgD, and IgA) or subclass, and may be derived from any suitable species. In some embodiments, the antibody is derived from human or murine. The antibody may also be a fully human antibody, humanized antibody or chimeric antibody prepared by recombinant methods.

As used herein, the term “monoclonal antibody (mAb)” refers to an antibody obtained from a substantially homogeneous population of antibodies, i.e., the individual antibodies constituting the population are identical except for possible naturally occurring mutations that may be present in minor amounts. Monoclonal antibodies are highly specific for same antigenic site(s), e.g., one epitope (monospecific), two epitopes (bispecific). The modifier “monoclonal” indicates the character of the antibody as being obtained from a substantially homogeneous population of antibodies, and is not to be construed as requiring some particular methods to produce the antibody.

An intact antibody or full-length antibody essentially comprises the antigen-binding variable region(s) as well as the light chain constant region(s) (CL) and heavy chain constant region(s) (CH), which could include CH1, CH2, CH3 and CH4, depending on the subtype of the antibody. An antigen-biding variable region (also known as a fragment variable region, Fv fragment) typically comprises a light chain variable region (V_{L}) and a heavy chain variable region (V_{H}). A constant region can be a constant region with a native sequence (such as a constant region with a human native sequence) or an amino acid sequence variant thereof. The variable region recognizes and interacts with the target antigen. The constant region can be recognized by and interacts with the immune system.

An antibody fragment may comprise a portion of an intact antibody, preferably its antigen binding region or variable region. Examples of antibody fragments include Fab, Fab′, F(ab′)2, Fd fragment consisting of V_{H} and CH1 domains, Fv fragment, single-domain antibody (dAb) fragment, and isolated complementarity determining region (CDR). The Fab fragment is an antibody fragment obtained by papain digestion of a full-length immunoglobulin, or a fragment having the same structure produced by, for example, recombinant expression. A Fab fragment comprises a light chain (comprising a V_{L} and a CL) and another chain, wherein the said other chain comprises a variable domain of the heavy chain (V_{H}) and a constant region domain of the heavy chain (CH1). The F(ab′)2 fragment is an antibody fragment obtained by pepsin digestion of an immunoglobulin at pH 4.0-4.5, or a fragment having the same structure produced by, for example, recombinant expression. The F(ab′)2 fragment essentially comprises two Fab fragments, wherein each heavy chain portion comprises a few additional amino acids, including the cysteines that form disulfide bonds connecting the two fragments. A Fab′ fragment is a fragment comprising one half of a F(ab′)2 fragment (one heavy chain and one light chain). The antibody fragment may comprise a plurality of chains joined together, for example, via a disulfide bond and/or via a peptide linker. Examples of antibody fragments also include single-chain Fv (scFv), Fv, dsFv, diabody, Fd and Fd′ fragments, and other fragments, including modified fragments. An antibody fragment typically comprises at least or about 50 amino acids, and typically at least or about 200 amino acids.

Antibodies according to the present disclosure can be prepared using techniques well known in the art, such as the following techniques or a combination thereof: recombinant techniques, phage display techniques, synthetic techniques, or other techniques known in the art. For example, a genetically engineered recombinant antibody (or antibody mimic) can be expressed by a suitable culture system (e.g., E. coli or mammalian cells). The engineering can refer to, for example, the introduction of a ligase-specific recognition sequence at its terminals.

The term “drug resistance” refers to resistance to drug therapy in the targeted disease. Many factors may contribute to resistance to therapy in cancer, such as tumor burden (the size of the tumor, or number of cells in the case of hematological tumors) and growth kinetics, interpatient and intratumor heterogeneity, immunosuppressive cancer microenvironments, enhanced genomic instability and selective therapeutic pressure. In the setting of HER2 -positive breast cancer, the reasons for drug resistance may include up-regulation of HER2 expression, HER2 mutations, abnormal activation of HER2 downstream signaling pathways, deletion of tumor suppressor genes, cell cycle dysregulation, low infiltration of tumor immune cells. Current research to overcome this resistance involves selective inhibitors of PI3K, inhibition of the IGF1R signaling pathway, and CDK inhibitors.

Payload refers to a substance that inhibits or prevents the expression activity of a cell, cellular function, and/or causes destruction of cells. The payloads currently used in ADCs are more toxic than chemotherapeutic drugs. Examples of payloads include, but are not limited to, drugs that target the following targets: microtubule cytoskeleton, DNA, RNA, kinesin-mediated protein transport, regulation of apoptosis. The drug that targets microtubule cytoskeleton may be, for example, a microtubule-stabilizing agent or a tubulin polymerization inhibitor. Examples of microtubule-stabilizing agents include but are not limited to taxanes. Examples of tubulin polymerization inhibitors include but are not limited to maytansinoids, auristatins, vinblastines, colchicines, and dolastatins. The DNA-targeting drug can be, for example, a drug that directly disrupts the DNA structure or a topoisomerase inhibitor. Examples of drugs that directly disrupt DNA structure include but are not limited to DNA double strand breakers, DNA alkylating agents, DNA intercalators. The DNA double strand breakers can be, for example, an enediyne antibiotic, including but not limited to dynemicin, esperamicin, neocarzinostatin, uncialamycin, and the like. The DNA alkylating agent may be, for example, a DNA bis-alkylator (i.e. DNA-cross linker) or a DNA mono-alkylator. Examples of DNA alkylating agents include but are not limited to pyrrolo[2,1-c][1,4]benzodiazepine (PBD) dimer, 1-(chloromethyl)-2,3-dihydrogen-1H-benzo[e]indole (CBI) dimer, CBI-PBD heterodimer, dihydroindolobenzodiazepine (IGN) dimer, duocarmycin-like compound, and the like. Examples of topoisomerase inhibitors include but are not limited to exatecan and derivatives thereof (such as DX8951f, DXd-(1) and DXd-(2), the structures of which are depicted below), camptothecins and anthracyclines. The RNA-targeting drug may be, for example, a drug that inhibits splicing, and examples thereof include but are not limited to pladienolide. Drugs that target kinesin-mediated protein transport can be, for example, mitotic kinesin inhibitors including, but not limited to, kinesin spindle protein (KSP) inhibitors.

HER2 refers to human epidermal growth factor receptor-2, which belongs to the epidermal growth factor (EGFR) receptor tyrosine kinase family (HER family, or EGFR family).

HER family proteins, which are type I transmembrane receptor tyrosine kinase (RTKs), have four members: HER1 (epidermal growth factor receptor (EGFR), ErbB1), HER2 (ErbB2), HER3 (ErbB3), and HER4 (ErbB4). The expression status of HER2 can be determined using known methods such as immunohistochemistry (IHC) and fluorescence in situ hybridization (FISH).

Trastuzumab is anti-HER2 IgG1 kappa monoclonal antibody which binds to the fourth extracellular domain (ECD4) of HER2. In addition to triggering antibody-dependent cellular cytotoxicity, trastuzumab also inhibits HER2 activation by inhibiting the cleavage of its extracellular domain, thus preventing the formation of the truncated and active form p95HER2. Trastuzumab (Herceptin®) is approved for the treatment of HER2-overexpressing breast cancer and metastatic gastric or gastroesophageal junction adenocarcinoma. In addition to its direct antitumor effect, trastuzumab has been found to have immune system-mediated antitumor activity.

Trastuzumab induces a sustained down-regulation of membrane-bound HER2. Long-term treatment of trastuzumab downregulates HER2. Expression of truncated HER2 without ECD, persistent activation of the PI3K/AKT signaling pathway, and hyperactivation of other tyrosine kinase receptors which compensates the oncogenic machinery may contribute to trastuzumab resistance. As demonstrated by gene amplification and RNA/protein overexpression, tumor cell with acquired resistance to trastuzumab continues to depend on the HER2 oncogene.

Pertuzumab binds to the extracellular dimerization domain (the second extracellular domain, ECD2) of HER2. In addition to triggering antibody-dependent cellular cytotoxicity, pertuzumab also prevent ligand-initiated heterodimerization of HER2 and other HER family members, especially HER3. Pertuzumab is therefore also known as a HER2 dimerization inhibitor. Pertuzumab (Perjeta®) is approved for the treatment of HER2-positive breast cancer in combination with trastuzumab and docetaxel, or in combination with trastuzumab and chemotherapy, or as monotherapy for adjuvant treatment of patients with HER2-positive early breast cancer at high risk of recurrence.

HER2-targeted ADCs are ADCs with HER2-targeted antibody as the targeting molecule. Current HER2-targeted ADCs on the market such as Disitamab Vedotin (RC48), T-DM1 (Kadcyla®), trastuzumab deruxtecan (T-DXd, Enhertu®) are used for treating HER2-positive cancers. T-DM1 contains trastuzumab, covalently linked to a microtubule inhibitory drug DM1 (a maytansine derivative) via a thiosuccinimide linker MCC (4-[N-maleimidomethyl] cyclohexane1-carboxylate). ADC drug GQ1001 developed by GeneQuantum contains trastuzumab, covalently linked to DM1 via a stable liker comprising open-ring thiosuccinimide structure.

As used herein, the term “antibody-drug conjugate” is also referred to as “conjugate”.

The term “tyrosine kinase inhibitor (TKI)” refers to a small molecule which can inhibit the tyrosine kinase activities of the targeted kinase. The terms “EGFR family tyrosine kinase inhibitor”, “HER family tyrosine kinase inhibitor” or “ErbB receptor tyrosine kinase inhibitor” (herein also as “EGFR family inhibitor”, “HER family inhibitor” or “ErbB receptor inhibitor” respectively for short) refers to a TKI which can inhibit the tyrosine kinase activities of one or more of the EGFR family (HER family) proteins. A HER2 inhibitor refers to a TKI which can inhibit the tyrosine kinase activities of HER2, but without excluding the possibility of inhibiting the tyrosine kinase activities of one or more of other HER family proteins. A HER2-selective inhibitor refers to a TKI which selectively inhibits HER2. Current HER family TKIs on the market include among others the HER2-selective inhibitors such as tucatinib and DZD1516, and pan-HER inhibitors such as afatinib, neratinib and pyrotinib, as well as lapatinib which is an EGFR/HER2 dual inhibitor. In monogenic tumors, resistance to tyrosine kinase inhibitors (TKIs) is frequently driven by gatekeeper mutations and other driver mutations, which maintain oncogene dependence.

Types of targeted therapy include, but are not limited to: enzyme inhibitors, enzyme activators, receptor modulators, ion channel modulators, transmembrane ion transport modulators, antibodies, antibody fragments, binding proteins for a given antigen, antibody mimics, and scaffold proteins having affinity for a given target. The term “small molecules for targeted therapy” or “small-molecule targeted therapy” refers to targeted therapies using small molecule compounds, and in some embodiments particularly refers to enzyme inhibitors, especially kinase inhibitors. The term “targeted therapy agent” or “targeted agent” refers to an agent which can be used as targeted therapy.

Examples of small-molecule targeted therapy include but are not limited to kinase inhibitors, such as antisecretory agents (e.g., brefeldin); 4-1BB agonist (e.g., PF-05082566); HSP90 inhibitors; checkpoint inhibitors; cell cycle inhibitors and differentiation inducers; caspase activators; tyrosine kinase inhibitors including but not limited to ALK inhibitors (e.g., crizotinib, ceritinib, alectinib, brigatinib, lorlatinib, entrectinib, belizatinib, repotrectinib, ensartinib), c-Met inhibitors (e.g., crizotinib, cabozantinib, capmatinib, tepotinib, foretinib, glesatinib, tivantinib, volitinib (savolitinib), bozitinib, ningetinib, glumetinib, kanitinib), HER family inhibitors (e.g., gefitinib, erlotinib, icotinib, afatinib, dacomitinib, osimertinib, almonertinib, lapatinib, neratinib (HKI-272), tucatinib (irbinitinib, ONT-380), ZN-A-1041, DZD1516, olmutinib, avitinib, pelitinib, furmonertinib (alflutinib)), FLT3 inhibitors (e.g., tandutinib, lestaurtinib, pexidartinib, gilteritinib, quizartinib, ponatinib, cabozantinib), VEGF/VEGFR/FGFR/PDGFR inhibitors (e.g., sorafenib, lenvatinib, regorafenib, nintedanib, apatinib, anlotinib, fruquintinib, erdafitinib, pemigatinib, avapritinib, ripretinib, cediranib, dovitinib, motesanib, sulfatinib, crenolanib, lucitanib, axitinib), RET inhibitors (e.g., vandetanib, selpercatinib, pralsetinib), tropomyosin receptor kinase (TRK) inhibitors (e.g., larotrectinib, entrectinib, cabozantinib, merestinib, belizatinib, sitravatinib, altiratinib, selitrectinib), Bcr-Abl1 inhibitors (e.g., imatinib, dasatinib, bosutinib, radotinib, nilotinib, ponatinib, asciminib, rebastinib, bafetinib), BTK inhibitors (e.g., ibrutinib, acalabrutinib, zanubrutinib, tirabrutinib, spebrutinib, evobrutinib, branebrutinib, HM71224, vecabrutinib, LOXO-305, fenebrutinib, ARQ-531), JAK inhibitors (e.g., ruxolitinib, fedratinib, tofacitinib, baricitinib, gandotinib, lestaurtinib, pacritinib); serine/threonine kinase inhibitors including but not limited to BRAF/MEK/ERK inhibitors (e.g., vemurafenib, dabrafenib, encorafenib, sorafenib, trametinib, binimetinib, cobimetinib, selumetinib, ravoxertinib), CDK inhibitors (e.g., palbociclib, ribociclib, abemaciclib, trilaciclib (G1T28)), PI3K/AKT/mTOR inhibitors (e.g., copanlisib, duvelisib, alpelisib (BYL719), serabelisib, bimiralisib, dactolisib (BEZ235), GDC-0084 (RG7666), GDC-0941, gedatolisib, ipatasertib, capivasertib, afuresertib, uprosertib, temsirolimus, sapanisertib (TAK-228), vistausertib (AZD2014), CC-223, BI860585, DS-3078a, ME-344, taselisib (GDC-0032), GDC-0349, OSI-027, P529); epigenetic inhibitors including but not limited to EZH2 inhibitors (e.g., 3-deazaneplanocin A (DZNep), EI1, EPZ005687, GSK126, GSK343, UNC1999, tazemetostat (EPZ6438), SHR2554, CPI-1205, DS-3201, PF-06821497, HH2853, EED226 (MAK683), BR-001, UNC6852, GNA002), HDAC inhibitors (e.g., TSA, vorinostat (SAHA), belinostat, panobinostat, romidepsin, tucidinostat (chidamide), valproic acid, phenylbutyrate, nicotinamide, EX-527, sirtinol, cambinol, entinostat), IDH1/2 inhibitors (e.g., enasidenib (AG-221), ivosidenib (AG-120), vorasidenib (AG-881)), DNMT inhibitors (e.g., azacytidine, decitabine), DOTIL methyltransferase inhibitors (e.g., pinometostat), BET inhibitors (e.g., birabresib, molibresib, ZEN003694, PLX51107), LSD1 inhibitors (e.g., iadademstat, INCB059872, CC-90011); BCL-2 inhibitors (e.g., oblimersen, ABT-737, ABT-263 (navitoclax), venetoclax, S55746 (also known as BCL201 or Servier-1), S64315 (MIK665), AZD-5991520, AMG-176, WEHI-539, A-1331852); hedgehog pathway inhibitors including but not limited to SMO inhibitors (e.g., vismodegib, sonidegib, glasdegib), GLI inhibitors (e.g., arsenic trioxide (ATO)), and itraconazole; proteasome inhibitors including but not limited to bortezomib, carfilzomib, ixazomib, marizomib (salinosporamide A), oprozomib, and delanzomib; PARP inhibitors including but not limited to nicotinamide, olaparib, rucaparib, niraparib, talazoparib, pamiparib, INO-1001, E7449, iniparib, AZD2461, amelparib, IMP4297, RBN-2397, fluzoparib, and veliparib (ABT-888).

The term “chemotherapeutic agent” refers to a chemical compound with selective toxicity and suitable therapeutic index and thus useful in the treatment of cancer. Types of chemotherapeutic agents include, but are not limited to: alkylating agents, antimetabolites, mitochondrial dysfunction inducers, cytotoxic/antitumor antibiotics, hormones and hormone analogs, photosensitizers, and drugs that target the following targets: microtubule cytoskeleton, DNA, RNA, kinesin-mediated protein transport, regulation of apoptosis. The drug that targets microtubule cytoskeleton may be, for example, a microtubule-stabilizing agent or a tubulin polymerization inhibitor. Examples of microtubule-stabilizing agents include but are not limited to taxanes (e.g., docetaxel, paclitaxel, nab-paclitaxel). Examples of tubulin polymerization inhibitors include but are not limited to maytansinoids, auristatins, vinblastines, colchicines, and dolastatins. The DNA-targeting drug can be, for example, a drug that directly disrupts the DNA structure or a topoisomerase inhibitor. Examples of drugs that directly disrupt DNA structure include but are not limited to DNA double strand breakers, DNA alkylating agents, DNA intercalators. The DNA double strand breakers can be, for example, an enediyne antibiotic, including but not limited to dynemicin, esperamicin, neocarzinostatin, uncialamycin. The DNA alkylating agent may be, for example, a DNA bis-alkylator (i.e. DNA-cross linker) or a DNA mono-alkylator. Examples of DNA alkylating agents include but are not limited to pyrrolo[2,1-c][1,4]benzodiazepine (PBD) dimer, 1-(chloromethyl)-2,3-dihydrogen-1H-benzo[e]indole (CBI) dimer, CBI-PBD heterodimer, dihydroindolobenzodiazepine (IGN) dimer, duocarmycin-like compound. Examples of DNA intercalators include but are not limited to platinum agents, ruthenium agents, rhodium agents, and organic bis-intercalators. Examples of topoisomerase inhibitors include but are not limited to camptothecins and anthracyclines. The RNA-targeting drug may be, for example, a drug that inhibits splicing, and examples thereof include but are not limited to pladienolide. Drugs that target kinesin-mediated protein transport can be, for example, mitotic kinesin inhibitors including, but not limited to, kinesin spindle protein (KSP) inhibitors. Other examples of chemotherapeutic agents include, but are not limited to: epothilones, combretastatin A-4 phosphate, combretastatin A-4 and derivatives thereof, indol-sulfonamides, dolastatin 10 and analogues, halichondrin B, eribulin, indole-3-oxoacetamide, podophyllotoxins, 7-diethylamino-3-(2′-benzoxazolyl)-coumarin (DBC), discodermolide, laulimalide, mitoxantrone, mitoguazone, nitrogen mustards, cyclophosphamide, nitrosoureasm, aziridines, benzodopa, carboquone, meturedepa, uredepa, aclacinomycin, actinomycin, antramycin, bleomycins, actinomycin C, carabicin, carminomycin, cardinophyllin, actinomycin D, daunorubicin, detorubicin, adriamycin, epirubicin, esorubicin, idarubicin, marcellomycin, mitomycins, nogalamycin, olivomycin, peplomycin, porfiromycin, puromycin, ferric adriamycin, rodorubicin, rufocromomycin, streptozocin, zinostatin, zorubicin, trichothecene, T-2 toxin, verracurin A, bacillocporin A, anguidine, ubenimex, azaserine, 6-diazo-5-oxo-L-norleucine, dimethyl folic acid, methotrexate, pteropterin, trimetrexate, edatrexate, fludarabine, 6-mercaptopurine, tiamiprine, thioguanine, cytarabine, capecitabine, ancitabine, gemcitabine, enocitabine, azacitidine, 6-azauridine, carmofur, dideoxyuridine, doxifluridine, floxuridine, calusterone, dromostanolone propionate, epitiostanol, mepitiostane, testolactone. The term “chemotherapy” refers to a therapy using one or more chemotherapeutic agents, especially a therapy using one chemotherapeutic agent. In some embodiments “chemotherapy” is used interchangeable with the term “chemotherapeutic agent”.

The terms “endocrine therapy (ET)” and “antihormone therapy” refers to therapies aiming to reduce the release of a hormone or the effect of a hormone on the growth of cancer cells. Endocrine therapy is typically used to treat breast, prostate and thyroid cancers. Examples of endocrine therapy agents (endocrine agents) include, but are not limited to: estrogen receptor modulators (e.g., tamoxifen, clomiphene, raloxifene, toremifene, lasofoxifene, arzoxifene, ospemifene, afimoxifene, arzoxifene, bazodoxifene), aromatase inhibitors (e.g., anastrozole, letrozole, exemestane), anti-adrenals (e.g., aminoglutethimide, mitotane, trilostane), androgen receptor modulators (e.g., flutamide, nilutamide, bicalutamide, enzalutamide, abiraterone, leuprorelin acetate, galeterone, goserelin).

The term “immune agonist” refers to an agonist which can induce or enhance immune response to the tumor, such as through activation of immune cells, including but not limited to DCs, B cells, macrophages, NK cells, and T cells. Examples of immune agonists include but are not limited to TLR agonists, such as agonists of TLR7 and/or TLR8 and/or TLR9 (e.g., imiquimod, resiquimod, 852A and VTX-2337) and STING agonists (e.g., ADU-S100 and MK-1454).

The terms “small-molecule targeted therapy” and “immune agonist”, and the terms “small-molecule targeted therapy” and “endocrine therapy”, and the terms “small-molecule targeted therapy” and chemotherapeutic agent” are not mutually exclusive as referred to herein.

A small molecule compound refers to a molecule with a size comparable to that of an organic molecule commonly used in medicine. The term does not encompass biological macromolecules (e.g., proteins, nucleic acids, etc.), but encompasses low molecular weight peptides or derivatives thereof, such as dipeptides, tripeptides, tetrapeptides, pentapeptides. Typically, the molecular weight of the small molecule compound can be, for example, about 100 to about 2000 Da, about 200 to about 1000 Da, about 200 to about 900 Da, about 200 to about 800 Da, about 200 to about 700 Da, about 200 to about 600 Da, about 200 to about 500 Da.

Cell proliferative disorders include but are not limited to tumors, cancers, and neoplastic tissue, including pre-malignant and non-neoplastic stages, and also include psoriasis, endometriosis, polyps and fibroadenoma.

The terms “cancer” and “cancerous” refer to the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer (e.g., gastric or gastroesophageal junction cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

The term “tumor”, as used herein, refers to all neoplastic cell growth and proliferation, whether malignant or benign, and all pre-cancerous and cancerous cells and tissues. A “cancer” or “cancer tissue” can include a tumor.

The terms “cancer”, “cancerous”, “cell proliferative disorder”, “proliferative disorder” and “tumor” are not mutually exclusive as referred to herein.

The terms “cancer therapeutic agents”, “antineoplastic agents”, “antimitotic agents” refers to agents with anti-cancer effect. The term “anti-cancer effect” as used herein, refers to a biological effect that can present as a reduction in the tumor burden, such as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-cancer effect can also refer to the prevention of the occurrence of a cancer, e.g., treatment effect on pre-cancerous lesions.

Examples of precancerous lesions include but are not limited to, actinic keratosis (solar keratosis), moles (dysplastic nevi), acitinic chelitis (farmer's lip), cutaneous horns, Barrett's esophagus, atrophic gastritis, dyskeratosis congenita, sideropenic dysphagia, lichen planus, oral submucous fibrosis, actinic (solar) elastosis and cervical dysplasia.

The term “wild-type” as used herein is understood in the art and refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild-type polypeptide or polynucleotide, respectively. Included in the term mutant is Single Nucleotide Polymorphism (SNP) where a single base pair distinction exists in the sequence of a nucleic acid strand compared to the most prevalently found (wild-type) nucleic acid strand. Cancers that are either associated with wild-type (Uniprot Accession: P04626) or mutant for HER2 or have amplification of ErbB2 (Genbank Accession X03363) or have over expression of HER2 protein are identified by known methods.

In some embodiments, the mutant form of HER2 comprises one or more of the following: insertions in exon 20 of HER2, deletions around amino acid residues 755-759 of HER2, one or more of the amino acid substitutions: D277G, D277H, D277V, D277Y, G309E, G309A, S310F, S310Y, S653C, V664E, R678Q, V697L, L726F, K753E, L755S, L755P, I767M, L768S, D769H, D769Y, V773L, A775_G776insYVMA, G776delinsVC, V777L, P780_Y781insGSP, T798I, T798M, C805S, V842I, T862A, L866M, L869R and R896C, as well as previously reported identical non-synonymous putative activating mutations (or indels) in COSMIC database or in prior art reference found in two or more unique specimens. In some embodiments, the mutant form of HER2 comprises one or more of the amino acid substitutions: D277G, D277H, D277V, D277Y, G309E, S310F, S310Y, S653C, R678Q, V697L, L726F, K753E, L755S, I767M, D769H, D769Y, A775_G776insYVMA, G776delinsVC, V777L, P780_Y781insGSP, T798I, T798M, V842I, L866M and L869R. In some embodiments, the mutant form of HER2 comprises one or more of the amino acid substitutions: S310F, S310Y, V697L, L726F, K753E, L755S, I767M, D769H, D769Y, V777L, P780_Y781insGSP, T798I, T798M, V842I and L869R. In some embodiments, the mutant form of HER2 comprises one or more of the amino acid substitutions: exon 20 insertions/deletions, L755S, V777L, T798I and V842I. In some embodiments, the mutant form of HER2 comprises one or more of the amino acid substitutions: S310F, S310Y, R678Q, D769H, D769Y and V777L.

“HER2-mutated” cancer includes but is not limited to cancer characterized by over expression of mutant HER2 protein.

The term “pharmaceutically acceptable” refers to that when contacted with the patient's tissue within the scope of normal medical judgment, no undue toxicity, irritation, allergic reactions, etc. shall arise.

The pharmaceutically acceptable salts of the additional cancer therapeutic agent include acid addition salts and base addition salts thereof. The method for preparing the pharmaceutically acceptable salt of the compound of the present disclosure is known to those skilled in the art. In an embodiment, the pharmaceutically acceptable salt is maleate.

The additional cancer therapeutic agent may exist in specific geometric or stereoisomeric forms, including cis and trans isomers, (−)- and (+)-enantiomers, (R)- and(S)-enantiomers, diastereomers, (D)-isomers, (L)-isomers, and their racemic mixtures and other mixtures, for example, mixtures enriched in enantiomers or diastereomers, all of which are within the scope of the present disclosure.

The solvates of a compound are forms wherein the compound of the present disclosure contains a solvent as a structural element of the compound crystal lattice, especially for example water, methanol, or ethanol. The amounts of solvents, especially water, can be present in stoichiometric or non-stoichiometric ratios.

The metabolites of a compound are substances formed in the body when the compounds of the present disclosure are administered. Such products can be produced, for example, by oxidation, reduction, hydrolysis, amidation, deamidation, esterification, enzymatic hydrolysis, etc. of the administered compound.

The prodrugs of a compound are certain derivatives of the compound that have less or no pharmacological activity themselves but when administered to or on the body, can be converted into the compound of the present disclosure with the desired activity by, for example, hydrolytic cleavage.

The term “polymorphism” or “polymorph” refers to a single polymorph or a mixture of more than one polymorph in any ratio.

The term “crystal form” or “crystal” refers to any solid substance exhibiting a three-dimensional order, as opposed to an amorphous solid substance, which produces a characteristic X-ray powder diffraction pattern with sharp and defined peaks.

The term “amorphous” refers to any solid substance which lacks order in three dimensions.

The term “effective amount” refers to those of the agents after administration will relieve one or more symptoms of the diseases to be treated to some extent.

The term “subject” as used herein in reference to individuals suffering from a disorder or a condition, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs and cats; laboratory animals including rodents, such as rats, mice and guinea pigs. Examples of non-mammals include, but are not limited to, birds and fish. In some embodiments of the method and use provided herein, the subject is a human.

### Pharmaceutical Combination

In an aspect, provided is a pharmaceutical combination, comprising the following (a) and (b);
(a) one or more conjugates having the structure of formula (I-1), (I-2), (I-3) and/or (I-4)

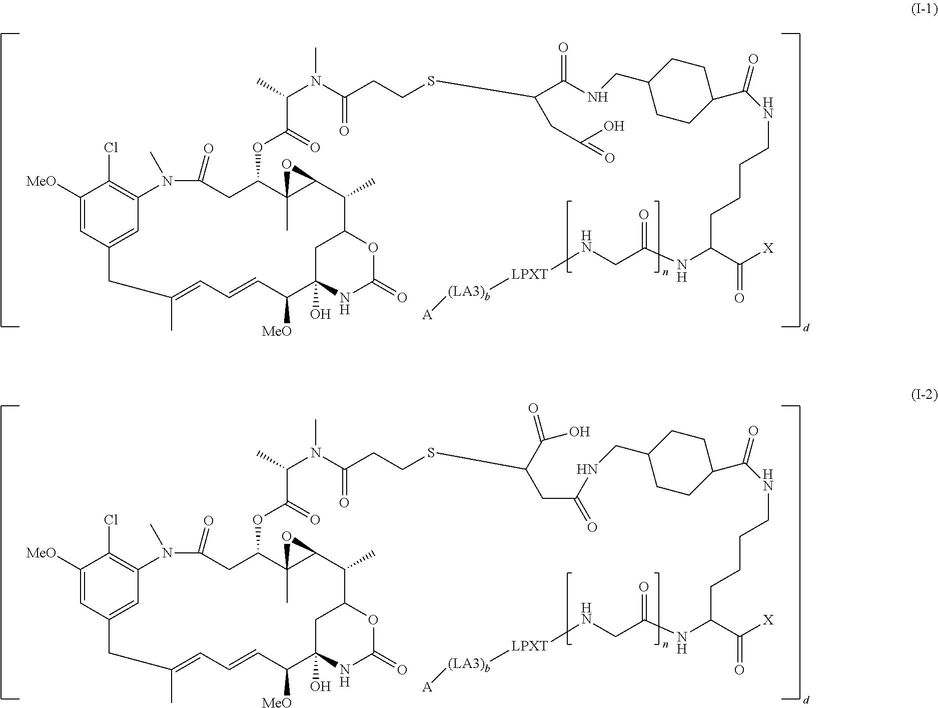

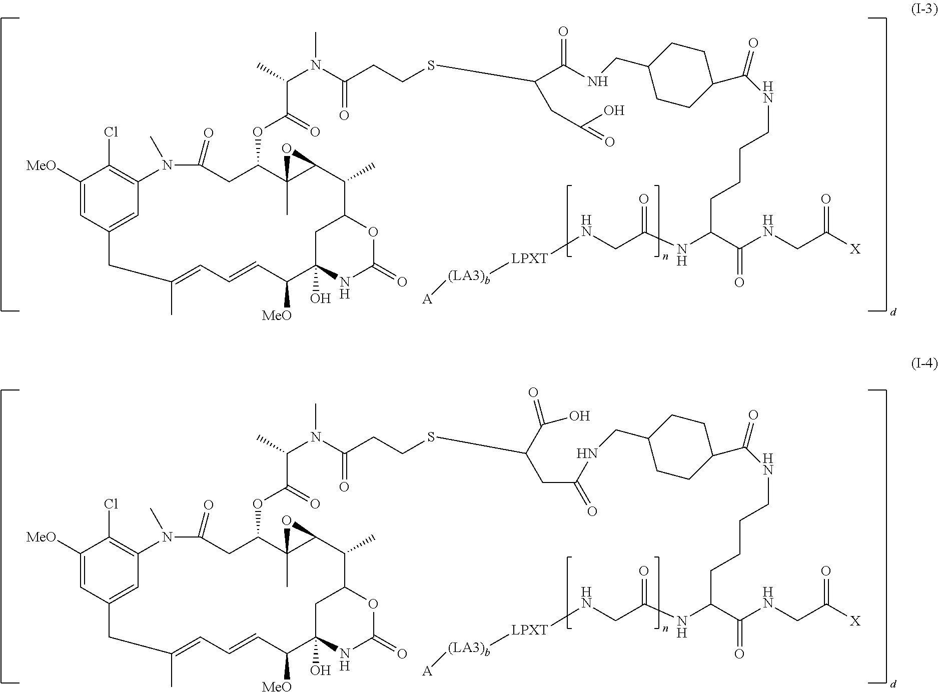

wherein n is an integer of 1 to 100, d is an integer of 1 to 20, A is an antibody or an antigen binding fragment thereof, LA3 comprises 1 to 100 series-connected structure units which are selected from the group consisting of one or more glycine and alanine; b is independently 0 or 1, that is, LA3 is independently present or absent, X in ligase recognition sequence LPXT is glutamic acid (E) or any other natural/unnatural amino acid, with L being leucine, P being proline, and T being threonine; x is —OH or —NH₂ group;
(b) one or more additional cancer therapeutic agents selected from the group consisting of: tyrosine kinase inhibitors, taxanes, and pyrimidine antimetabolites (also known as pyrimidine analogues).

In an embodiment, the ligase recognition sequence LPXT has the amino acid sequence of SEQ ID No: 11.

The additional cancer therapeutic agent of the present disclosure encompass the pharmaceutically acceptable salts, stereoisomers, solvates (preferably hydrates), polymorphs, tautomers, isotopic compounds, metabolites or prodrugs thereof.

### Specific Embodiments of the Pharmaceutical Combination

Further embodiments of the pharmaceutical combination of the present disclosure are set forth below by describing both the item (a) and the item (b), or by specifying any components of the item (a) and/or the item (b), or other components of the pharmaceutical combination. It should be understood that any pharmaceutical combination formed by combining any mutually nonexclusive embodiments disclosed herein are specifically revealed herein, regardless of whether the said pharmaceutical combination is explicitly recited.

### Item (a): Conjugate

In an embodiment, the conjugate of formula (I-1) (1-2) (I-3) or (I-4) is an antibody-drug conjugate formed by covalently connecting the following parts: the payload, the linker and the antibody; wherein the payload is DM-1 (a maytansine derivative); the linker is a stable linker which does not tend to undergo reverse Michael addition or exchange with other thiol group. In an embodiment, the antibody is connected to the rest moiety of the conjugate by one or more connections form by ligase-catalyzed site-specific conjugation.

In an embodiment, the conjugate has the structure of formula (I-1) and/or (I-2). In an embodiment, the conjugate has the structure of formula (I-3) and/or (I-4).

In an embodiment, n is an integer of 3 to 10, preferably 2 to 5, particularly 3. In an embodiment, d is an integer of 1 to 10, preferably 1 to 4, more preferably 1 or 2, particularly 2. In an embodiment, LA3 is GGGGS, GGGGSGGGGS, GGGGGGGGSGGGGS, or GA; preferably, LA3 is GA. In an embodiment, b is independently 0 or 1; preferably, b is 1, that is, LA3 is present. In an embodiment, X in ligase recognition sequence LPXT is glutamine (Q) or glutamic acid (E); preferably glutamic acid (E). In an embodiment, x is —NH₂ group.

In an embodiment, the antibody is an anti-CD19 antibody, anti-CD20 antibody, anti-CD22 antibody, anti-CD25 antibody, anti-CD30/TNFRSF8 antibody, anti-CD33 antibody, anti-CD37 antibody, anti-CD44v6 antibody, anti-CD56 antibody, anti-CD70 antibody, anti-CD71 antibody, anti-CD74 antibody, anti-CD79b antibody, anti-CD117/KITk antibody, anti-CD123 antibody, anti-CD138 antibody, antibody, anti-CD174 antibody, anti-CD227/MUC1 antibody, anti-CD352 antibody, anti-CLDN18.2 antibody, anti-DLL3 antibody, anti-ErbB2/HER2 antibody, anti-CN33 antibody, anti-GPNMB antibody, anti-ENPP3 antibody, anti-Nectin-4 antibody, anti-EGFRvIII antibody, anti-SLC44A4/AGS-5 antibody, anti-CEACAM5 antibody, anti-PSMA antibody, anti-TIM1 antibody, anti-LY6E antibody, anti-LIV1 antibody, anti-Nectin4 antibody, anti-SLITRK6 antibody, anti-HGFR/cMet antibody, anti-SLAMF7/CS1 antibody, anti-EGFR antibody, anti-BCMA antibody, anti-AXL antibody, anti-NaPi2B antibody, anti-GCC antibody, anti-STEAP1 antibody, anti-MUC16 antibody, anti-Mesothelin antibody, anti-ETBR antibody, anti-EphA2 antibody, anti-5T4 antibody, anti-FOLR1 antibody, anti-LAMP1 antibody, anti-Cadherin 6 antibody, anti-FGFR2 antibody, anti-FGFR3 antibody, anti-CA6 antibody, anti-CanAg antibody, anti-integrin αV antibody, anti-TDGF1 antibody, anti-Ephrin A4 antibody, anti-TROP2 antibody, anti-PTK7 antibody, anti-NOTCH3 antibody, anti-C4.4A antibody, anti-FLT3 antibody, anti-B7H3/4 antibody, anti-Tissue Factor antibody, or anti-ROR1/2 antibody; preferably anti-HER2 antibody, anti-FGFR3 antibody, anti-Trop2 antibody, anti-HER3 antibody or anti-FRα antibody.

In a preferred embodiment, the antibody is anti-HER2 antibody. In an embodiment, the antibody is an anti-human HER2 antibody. Examples of anti-human HER2 antibodies include but are not limited to Pertuzumab and Trastuzumab.

In one embodiment, the antibody is an anti-HER2 antibody or antigen-binding fragment thereof, comprising a light chain variable region (V_{L}) and a heavy chain variable region (V_{H}), wherein the V_{L} comprises LCDR1 comprising the amino acid sequence of SEQ ID NO: 1, LCDR2 comprising the amino acid sequence of SEQ ID NO: 2, LCDR3 comprising the amino acid sequence of SEQ ID NO: 3, and wherein the V_{H} comprises HCDR1 comprising the amino acid sequence of SEQ ID NO: 4, HCDR2 comprising the amino acid sequence of SEQ ID NO: 5, HCDR3 comprising the amino acid sequence of SEQ ID NO: 6. In one embodiment, the CDR regions are defined by KABAT. In one embodiment, the antibody is an anti-HER2 antibody or antigen-binding fragment thereof, comprising a light chain variable region (V_{L}) and a heavy chain variable region (V_{H}), wherein the V_{L} has an amino acid sequence having at least about 85%, at least about 90% or at least about 95% sequence identity with SEQ ID NO: 7, and the V_{H} has an amino acid sequence having at least about 85%, at least about 90% or at least about 95% sequence identity with SEQ ID NO: 8. In a preferred embodiment, the anti-HER2 antibody or antigen-binding fragment thereof comprises a light chain variable region (V_{L}) and a heavy chain variable region (V_{H}), wherein the V_{L} has the amino acid sequence of SEQ ID NO: 7, and the V_{H} has the amino acid sequence of SEQ ID NO: 8. In a particular embodiment, the anti-HER2 antibody comprises a light chain and a heavy chain, wherein the light chain has an amino acid sequence having at least about 85%, at least about 90% or at least about 95% sequence identity with SEQ ID NO: 9, and the heavy chain has an amino acid sequence having at least about 85%, at least about 90% or at least about 95% sequence identity with SEQ ID NO: 10. In a preferred embodiment, the anti-human HER2 antibody is selected from trastuzumab and engineered anti-HER2 antibodies based on trastuzumab. In an embodiment, the antibody is trastuzumab. In a preferred embodiment, the antibody is connected to the rest moiety of the conjugate via the C-terminal of one of or both of, preferably both of, the light chains of the antibody. In a specific embodiment, A in formula (I-1), (I-2), (I-3) and (I-4) is trastuzumab. In an embodiment, the conjugate of formula (I-1) has the structure of formula (i-1)

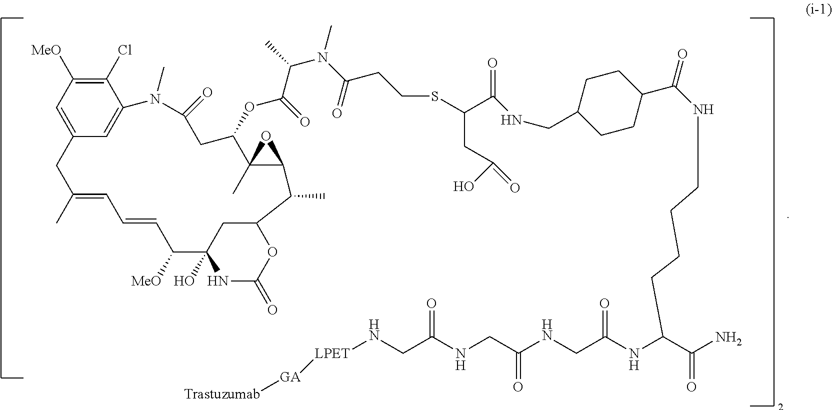

In an embodiment, the conjugate of formula (I-2) has the structure of formula (i-2)

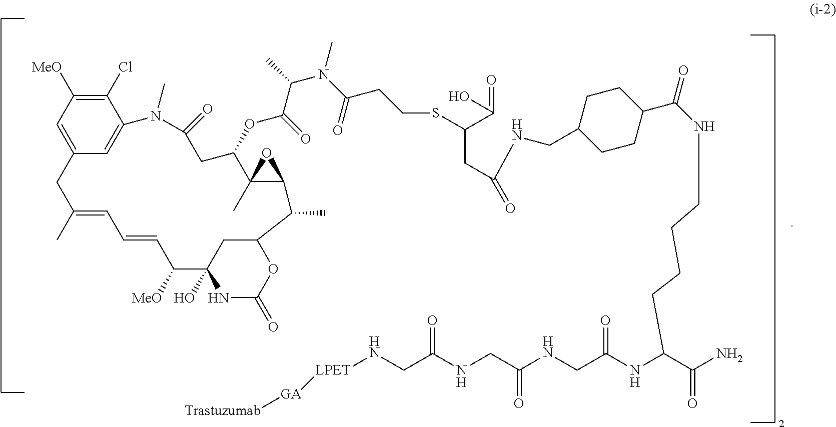

In an embodiment, the conjugate has the above structure of formula (i-1) and/or formula (i-2). In a preferred embodiment, trastuzumab is connected to the rest moiety of the conjugate via the C-terminal of both of the light chains of trastuzumab.

### Item (b): Additional Cancer Therapeutic Agent

In an embodiment, the additional cancer therapeutic agent is selected from small molecule compounds. In an embodiment, the additional cancer therapeutic agent is selected from the group consisting of: targeted therapy agents, chemotherapeutic agents, endocrine therapy agents and immune agonists.

In an embodiment, the additional cancer therapeutic agent is selected from the group consisting of: vaccines, antibodies, fusion proteins, HER2-targeted ADCs, fibrinolytic agents; antimigratory agents; antisecretory agents; HSP90 inhibitors; anti-angiogenic compounds; checkpoint inhibitors; cell cycle inhibitors and differentiation inducers; mitochondrial dysfunction inducers; caspase activators; kinase inhibitors, proteasome inhibitors, poly (ADP-ribose) polymerases (PARP) inhibitors, enhancer of zeste homolog 2 (EZH2) inhibitors, histone deacetylase (HDAC) inhibitors, isocitrate dehydrogenase 1/2 (IDH1/2) inhibitors, DNA methyltransferases (DNMT) inhibitors, DOT1 like histone lysine) (DOT1L) methyltransferase inhibitors, bromodomain and extraterminal domain proteins (BET) inhibitors, lysine-specific demethylase 1 (LSD1) inhibitors, B-cell lymphoma-2 (BCL-2) inhibitors, transmembrane transducer smoothened (SMO) inhibitors, GLI inhibitors, itraconazole, alkylating agents, antimetabolites, cytotoxic/antitumor antibiotics, hormones and hormone analogs, photosensitizers, microtubule-stabilizing agents, tubulin polymerization inhibitors, DNA double strand breakers, DNA alkylating agents, DNA intercalators, topoisomerase inhibitors, mitotic kinesin inhibitors, estrogen receptor modulators, aromatase inhibitors, anti-adrenals, androgen receptor modulators, toll-like receptor (TLR) agonists, and stimulator of interferon genes (STING) agonists.

In an embodiment, the additional cancer therapeutic agent is selected from the group consisting of: tyrosine kinase inhibitors, chemotherapeutic agents, and pyrimidine antimetabolites.

In an embodiment, the kinase inhibitors are selected from the group consisting of: tyrosine kinase inhibitors and serine/threonine kinase inhibitors. In an embodiment, the tyrosine kinase inhibitors are selected from the group consisting of: anaplastic lymphoma kinase (ALK) inhibitors, c-Met inhibitors, HER family inhibitors, FMS-like tyrosine kinase 3 (FLT3) inhibitors, vascular endothelial growth factor (VEGF)/vascular endothelial growth factor receptor (VEGFR), fibroblast growth factor receptor (FGFR) inhibitors, platelet-derived growth factor receptor (PDGFR) inhibitors, rearranged during transfection (RET) kinase inhibitors, tropomyosin receptor kinase (TRK) inhibitors, Bcr-Abl1 inhibitors, Bruton's tyrosine kinase (BTK) inhibitors, and Janus kinase (JAK) inhibitors. In an embodiment, the serine/threonine kinase inhibitors are selected from the group consisting of: BRAF inhibitors, mitogen-activated protein kinase (MEK) inhibitors, extracellular signal-regulated kinase (ERK) inhibitors, cyclin-dependent kinase (CDK) inhibitors, phosphoinositide 3-kinase (PI3K) inhibitors, AKT inhibitors, mammalian target of rapamycin (mTOR) inhibitors.

In an embodiment, the antibodies as additional cancer therapeutic agents are selected from the group consisting of: trastuzumab, pertuzumab, margetuximab, bevacizumab, atezolizumab, pembrolizumab, PF-05082566, tislelizumab, and their biosimilars (e.g., Herzuma® (trastuzumab-pkrb)).

In an embodiment, the additional cancer therapeutic agent is selected from the group consisting of: brefeldin, PF-05082566, crizotinib, ceritinib, alectinib, brigatinib, lorlatinib, entrectinib, belizatinib, repotrectinib, ensartinib, cabozantinib, capmatinib, tepotinib, foretinib, glesatinib, tivantinib, volitinib (savolitinib), bozitinib, ningetinib, glumetinib, kanitinib, gefitinib, erlotinib, icotinib, afatinib, dacomitinib, osimertinib, almonertinib, lapatinib, neratinib (HKI-272), tucatinib (irbinitinib, ONT-380), ZN-A-1041, DZD1516, olmutinib, avitinib, pelitinib, furmonertinib (alflutinib), tandutinib, lestaurtinib, pexidartinib, gilteritinib, quizartinib, ponatinib, cabozantinib, sorafenib, lenvatinib, regorafenib, nintedanib, apatinib, anlotinib, fruquintinib, erdafitinib, pemigatinib, avapritinib, ripretinib, cediranib, dovitinib, motesanib, sulfatinib, crenolanib, lucitanib, axitinib, vandetanib, selpercatinib, pralsetinib, larotrectinib, merestinib, sitravatinib, altiratinib, selitrectinib, imatinib, dasatinib, bosutinib, radotinib, nilotinib, asciminib, rebastinib, bafetinib, ibrutinib, acalabrutinib, zanubrutinib, tirabrutinib, spebrutinib, evobrutinib, branebrutinib, HM71224, vecabrutinib, LOXO-305, fenebrutinib, ARQ-531, ruxolitinib, fedratinib, tofacitinib, baricitinib, gandotinib, pacritinib, vemurafenib, dabrafenib, encorafenib, trametinib, binimetinib, cobimetinib, selumetinib, ravoxertinib, palbociclib, ribociclib, abemaciclib, trilaciclib (G1T28), copanlisib, duvelisib, alpelisib (BYL719), serabelisib, bimiralisib, dactolisib (BEZ235), GDC-0084 (RG7666), GDC-0941, gedatolisib, ipatasertib, capivasertib, afuresertib, uprosertib, temsirolimus, sapanisertib (TAK-228), vistausertib (AZD2014), CC-223, BI860585, DS-3078a, ME-344, taselisib (GDC-0032), GDC-0349, OSI-027, P529, 3-deazaneplanocin A (DZNep), EI1, EPZ005687, GSK126, GSK343, UNC1999, tazemetostat (EPZ6438), SHR2554, CPI-1205, DS-3201, PF-06821497, HH2853, EED226 (MAK683), BR-001, UNC6852, GNA002, TSA, vorinostat (SAHA), belinostat, panobinostat, romidepsin, tucidinostat (chidamide), valproic acid, phenylbutyrate, nicotinamide, EX-527, sirtinol, cambinol, entinostat, enasidenib (AG-221), ivosidenib (AG-120), vorasidenib (AG-881), azacytidine, decitabine, pinometostat, birabresib, molibresib, ZEN003694, PLX51107, iadademstat, INCB059872, CC-90011, oblimersen, ABT-737, ABT-263 (navitoclax), venetoclax, S55746 (also known as BCL201 or Servier-1), S64315 (MIK665), AZD-5991520, AMG-176, WEHI-539, A-1331852, vismodegib, sonidegib, glasdegib, arsenic trioxide (ATO), itraconazole, bortezomib, carfilzomib, ixazomib, marizomib (salinospor-amide A), oprozomib, delanzomib, olaparib, rucaparib, niraparib, talazoparib, pamiparib, INO-1001, E7449, iniparib, AZD2461, amelparib, IMP4297, RBN-2397, fluzoparib, veliparib (ABT-888), docetaxel, paclitaxel, nab-paclitaxel, maytansinoids, auristatins, vinblastines, colchicines, dolastatins, dynemicin, esperamicin, neocarzinostatin, uncialamycin, pyrrolo[2,1-c][1,4]benzodiazepine (PBD) dimer, 1-(chloromethyl)-2,3-dihydrogen-1H-benzo[e]indole (CBI) dimer, CBI-PBD heterodimer, dihydroindolobenzodiazepine (IGN) dimer, duocarmycin-like compound, platinum agents, ruthenium agents, rhodium agents, organic bis-intercalators, camptothecins, anthracyclines, pladienolide, epothilones, combretastatin A-4 phosphate, combretastatin A-4 and derivatives thereof, indol-sulfonamides, dolastatin 10 and analogues, halichondrin B, eribulin, indole-3-oxoacetamide, podophyllotoxins, 7-diethylamino-3-(2′-benzoxazolyl)-coumarin (DBC), discodermolide, laulimalide, mitoxantrone, mitoguazone, nitrogen mustards, cyclophosphamide, nitrosoureasm, aziridines, benzodopa, carboquone, meturedepa, uredepa, aclacinomycin, actinomycin, antramycin, bleomycins, actinomycin C, carabicin, carminomycin, cardinophyllin, actinomycin D, daunorubicin, detorubicin, adriamycin, epirubicin, esorubicin, idarubicin, marcellomycin, mitomycins, nogalamycin, olivomycin, peplomycin, porfiromycin, puromycin, ferric adriamycin, rodorubicin, rufocromomycin, streptozocin, zinostatin, zorubicin, trichothecene, T-2 toxin, verracurin A, bacillocporin A, anguidine, ubenimex, azaserine, 6-diazo-5-oxo-L-norleucine, dimethyl folic acid, methotrexate, pteropterin, trimetrexate, edatrexate, fludarabine, 6-mercaptopurine, tiamiprine, thioguanine, cytarabine, capecitabine, ancitabine, gemcitabine, enocitabine, azacitidine, 6-azauridine, carmofur, dideoxyuridine, doxifluridine, floxuridine, calusterone, dromostanolone propionate, epitiostanol, mepitiostane, testolactone, tamoxifen, clomiphene, raloxifene, toremifene, lasofoxifene, arzoxifene, ospemifene, afimoxifene, bazodoxifene, anastrozole, letrozole, exemestane, aminoglutethimide, mitotane, trilostane, flutamide, nilutamide, bicalutamide, enzalutamide, abiraterone, leuprorelin acetate, galeterone, goserelin, imiquimod, resiquimod, 852A, VTX-2337, ADU-S100 and MK-1454.

In an embodiment, the additional cancer therapeutic agent is selected from the group consisting of: kinase inhibitors and chemotherapeutic agents. In an embodiment, the additional cancer therapeutic agent is selected from the group consisting of: tyrosine kinase inhibitors, taxanes, and pyrimidine antimetabolites (also known as pyrimidine analogues).

In an embodiment, the additional cancer therapeutic agent is selected from the group consisting of kinase inhibitors. In an embodiment, the kinase inhibitors are selected from the HER family tyrosine kinase inhibitors. In a specific embodiment, the HER family tyrosine kinase inhibitor is selected from the group consisting of: lapatinib, tucatinib, afatinib, neratinib and pyrotinib. In another specific embodiment, the additional cancer therapeutic agent is selected from the group consisting of tucatinib, neratinib and pyrotinib; preferably tucatinib or pyrotinib. In another specific embodiment, the additional cancer therapeutic agent is pyrotinib. In an embodiment, the tyrosine kinase inhibitor is in the form of its pharmaceutically acceptable salt. In a particular embodiment, pyrotinib is in the form of pyrotinib maleate. In another specific embodiment, the HER family tyrosine kinase inhibitor is ZN-A-1041 or DZD1516.

In an embodiment, the additional cancer therapeutic agent is selected from taxanes. In an embodiment, the additional cancer therapeutic agent is selected from docetaxel and paclitaxel, preferably paclitaxel.

In an embodiment, the additional cancer therapeutic agent is selected from the group consisting of pyrimidine antimetabolites. In an embodiment, the pyrimidine antimetabolites are selected from the group consisting of: cytarabine, capecitabine, ancitabine, gemcitabine, enocitabine, carmofur, dideoxyuridine, doxifluridine and floxuridine. In another specific embodiment, the additional cancer therapeutic agent is capecitabine.

In an embodiment, the additional cancer therapeutic agent is selected from the group consisting of: tucatinib, neratinib, pyrotinib, paclitaxel and capecitabine.

In an embodiment, the antibody as additional cancer therapeutic agent is an anti-CD19 antibody, anti-CD20 antibody, anti-CD22 antibody, anti-CD25 antibody, anti-CD30/TNFRSF8 antibody, anti-CD33 antibody, anti-CD37 antibody, anti-CD44v6 antibody, anti-CD56 antibody, anti-CD70 antibody, anti-CD71 antibody, anti-CD74 antibody, anti-CD79b antibody, anti-CD117/KITk antibody, anti-CD123 antibody, anti-CD138 antibody, anti-CD142 antibody, anti-CD174 antibody, anti-CD227/MUC1 antibody, anti-CD352 antibody, anti-CLDN18.2 antibody, anti-DLL3 antibody, anti-ErbB2/HER2 antibody, anti-CN33 antibody, anti-GPNMB antibody, anti-ENPP3 antibody, anti-Nectin-4 antibody, anti-EGFRvIII antibody, anti-SLC44A4/AGS-5 antibody, anti-CEACAM5 antibody, anti-PSMA antibody, anti-TIM1 antibody, anti-LY6E antibody, anti-LIV1 antibody, anti-Nectin4 antibody, anti-SLITRK6 antibody, anti-HGFR/cMet antibody, anti-SLAMF7/CS1 antibody, anti-EGFR antibody, anti-BCMA antibody, anti-AXL antibody, anti-NaPi2B antibody, anti-GCC antibody, anti-STEAP1 antibody, anti-MUC16 antibody, anti-Mesothelin antibody, anti-ETBR antibody, anti-EphA2 antibody, anti-5T4 antibody, anti-FOLR1 antibody, anti-LAMP1 antibody, anti-Cadherin 6 antibody, anti-FGFR2 antibody, anti-FGFR3 antibody, anti-CA6 antibody, anti-CanAg antibody, anti-integrin αV antibody, anti-TDGF1 antibody, anti-Ephrin A4 antibody, anti-TROP2 antibody, anti-PTK7 antibody, anti-NOTCH3 antibody, anti-C4.4A antibody, anti-FLT3 antibody, anti-B7H3/4 antibody, anti-Tissue Factor antibody, or anti-ROR1/2 antibody; preferably anti-HER2 antibody, anti-FGFR3 antibody, anti-Trop2 antibody, anti-HER3 antibody or anti-FRα antibody.

In an embodiment, the additional cancer therapeutic agent is selected from the group consisting of: palbociclib, afatinib, poziotinib, metronomic temozolomide, PD-0332991, tucatinib, abemaciclib, neratinib, copanlisib, anthracycline-based chemotherapy, pertuzumab, BYL719, DZD1516, lapatinib, pembrolizumab, vinorelbine, capecitabine, atezolizumab, osimertinib, ribociclib, pyrotinib (e.g., pyrotinib maleate), venetoclax, GDC-0941, trastuzumab, 4-1BB agonist (e.g., PF-05082566), BN-Brachyury, TPIV100, M7824, taxane (e.g., docetaxel, paclitaxel, nab-paclitaxel such as Abraxane®), entinostat, vemurafenib, alectinib, ZN-A-1041, sargramostim, carboplatin, doxorubicin, epirubicin, cyclophosphamide, taselisib, eribulin, gemcitabine, tislelizumab, celecoxib, Herzuma®.

### Pharmaceutical Composition and Pharmaceutical Preparation

The pharmaceutical combination of the present disclosure can further comprise one or more pharmaceutically acceptable carriers. In an embodiment wherein the pharmaceutical combination is provided in a unique form such as a pharmaceutical composition or mixture, the compound or agent(s) contained therein are combined with the same pharmaceutically acceptable carriers, for simultaneous, separate or sequential use. Accordingly, provided is a pharmaceutical composition, comprising the pharmaceutical combination of the present disclosure.

The pharmaceutical composition of the present disclosure can be prepared in a manner known per se and are those suitable for enteral, such as oral or rectal, and parenteral administration to mammals (warm-blooded animals), including man, comprising a therapeutically effective amount of conjugate of formula (I-1), (I-2), (1-3), and/or (I-4) and at least a therapeutically effective amount of the additional cancer therapeutic agent, or further in combination with one or more pharmaceutically acceptable carries, especially suitable for enteral or parenteral application.

In an alternative embodiment, wherein the pharmaceutical combination is provided in separated forms such as different compartments in a kit or in different kits, the agents contained therein, either the conjugate of formula (I-1), (I-2), (1-3), and/or (I-4) or the additional cancer therapeutic agent(s), are independently combined with the pharmaceutically acceptable carriers. The pharmaceutically acceptable carriers for each of the agent may be identical or different according to practice requirement. Accordingly, provided is also a kit, comprising (a) a conjugate of formula (I-1), (I-2), (I-3), and/or (I-4) and optional one or more pharmaceutically acceptable carriers; (b) one or more additional cancer therapeutic agents and optional one or more pharmaceutically acceptable carriers; and (c) instruction for using (a) and (b). The conjugate of formula (I-1), (1-2), (1-3), and/or (I-4) and the additional cancer therapeutic agents are defined as above. Accordingly, provided is a kit, comprising the pharmaceutical combination of the present disclosure.

In an embodiment, the conjugate comprised in the kit is for administration in an amount ranging from about 4 mg/kg to about 10 mg/kg once every 18 days to every 28 days; for example about 4 mg/kg, about 5 mg/kg, about 6 mg/kg, about 7 mg/kg, about 7.2 mg/kg, about 8 mg/kg, about 8.4 mg/kg, about 9 mg/kg or about 10 mg/kg (or range between any two of these values) once every 18 days to every 28 days; about 4 mg/kg, about 5 mg/kg, about 6 mg/kg, about 7 mg/kg, about 8 mg/kg, about 9 mg/kg or about 10 mg/kg (or range between any two of these values) once every 18 days; or about 4 mg/kg, about 5 mg/kg, about 6 mg/kg, about 7 mg/kg, about 7.2 mg/kg, about 8 mg/kg, about 8.4 mg/kg, about 9 mg/kg or about 10 mg/kg (or range between any two of these values) once every 20 days; or about 4 mg/kg, about 5 mg/kg, about 6 mg/kg, about 7 mg/kg, about 7.2 mg/kg, about 8 mg/kg, about 8.4 mg/kg, about 9 mg/kg or about 10 mg/kg (or range between any two of these values) once every 21 days; or about 4 mg/kg, about 5 mg/kg, about 6 mg/kg, about 7 mg/kg, about 7.2 mg/kg, about 8 mg/kg, about 8.4 mg/kg, about 9 mg/kg or about 10 mg/kg (or range between any two of these values) once every 24 days; or about 4 mg/kg, about 5 mg/kg, about 6 mg/kg, about 7 mg/kg, about 7.2 mg/kg, about 8 mg/kg, about 8.4 mg/kg, about 9 mg/kg or about 10 mg/kg (or range between any two of these values) once every 28 days. In an embodiment, the conjugate comprised in the kit is for administration in an amount ranging from about 5 mg/kg to about 9 mg/kg once every 21 days. In an embodiment, the conjugate comprised in the kit is for administration in an amount ranging from about 6 mg/kg to about 8.4 mg/kg once every 21 days, for example, about 6 mg/kg, about 7.2 mg/kg, or about 8.4 mg/kg once every 21 days. In a very special embodiment, the conjugate comprised in the kit is for administration in an amount of about 7.2 mg/kg once every 21 days.

In an embodiment, the conjugate comprised in the kit is for administration in an amount ranging from about 200 mg to about 700 mg once every 18 days to every 28 days. In an embodiment, the conjugate comprised in the kit is for administration in an amount ranging from about 200 mg to about 700 mg once every 18 days. In an embodiment, the conjugate comprised in the kit is for administration in an amount ranging from about 200 mg to about 700 mg once every 21 days. In an embodiment, the conjugate comprised in the kit is for administration in an amount ranging from about 200 mg to about 700 mg once every 24 days. In an embodiment, the conjugate comprised in the kit is for administration in an amount ranging from about 200 mg to about 700 mg once every 28 days. In an embodiment, the amount of conjugate comprised in the kit is about 200 mg, about 260 mg, about 300 mg, about 360 mg, about 400 mg, about 452 mg, about 460 mg, about 500 mg, about 504 mg, about 560 mg, about 600 mg, about 630 mg, about 660 mg, about 700 mg (or range between any two of these values) once every 21 days. In an embodiment, the amount of conjugate comprised in the kit is about 360 mg, about 452 mg or about 504 mg once every 21 days.

In an embodiment, the conjugate comprises structure of formula (i-1) and/or formula (i-2). In some embodiment, the conjugate consists of structure of formula (i-1) and formula (i-2).

In an embodiment, the additional cancer therapeutic agent comprised in the kit is for administration in an amount ranging from about 100 mg to about 600 mg once daily for 18 days to 30 days per treatment cycle; for example, about 100 mg to about 500 mg, or about 200 mg to about 400 mg, or about 260 mg to about 400 mg, or about 300 mg to about 350 mg once daily for 18 days per treatment cycle; about 100 mg to about 500 mg, or about 200 mg to about 400 mg, or about 260 mg to about 400 mg, or about 300 mg to about 350 mg once daily for 21 days per treatment cycle; about 100 mg to about 500 mg, or about 200 mg to about 400 mg, or about 260 mg to about 400 mg, or about 300 mg to about 350 mg once daily for 24 days per treatment cycle; about 100 mg to about 500 mg, or about 200 mg to about 400 mg, or about 260 mg to about 400 mg, or about 300 mg to about 350 mg once daily for 28 days per treatment cycle; about 100 mg to about 500 mg, or about 200 mg to about 400 mg, or about 260 mg to about 400 mg, or about 300 mg to about 350 mg once daily for 30 days per treatment cycle; preferably from about 300 mg to about 350 mg once daily for 18 days to 30 days per treatment cycle; for example, about 300 mg, about 310 mg, about 315 mg, about 320 mg, about 326 mg, about 330 mg, about 340 mg or about 350 mg once daily for 18 days to 30 days per treatment cycle. In an embodiment, the additional cancer therapeutic agent comprised in the kit is for administration in an amount of about 320 mg once daily for 28 days per treatment cycle.

In an embodiment, the additional cancer therapeutic agent is tyrosine kinase inhibitors, taxanes, or pyrimidine antimetabolites. In an embodiment, the additional cancer therapeutic agent is tucatinib, neratinib, pyrotinib, paclitaxel or capecitabine.

In an embodiment, the cell proliferative disorder is HER2-positive breast cancer; preferably HER2-positive metastatic breast cancer; and/or
the subject in need of the kit has received prior treatment(s) selected from the group consisting of: trastuzumab, pertuzumab, pyrotinib, capecitabine, palbociclib, letrozole and combination thereof; and/or
the conjugate comprised in the kit is for administration in an amount of about 5 mg/kg to about 10 mg/kg once every 21 days; preferably about 6 mg/kg to about 8.4 mg/kg once every 21 days, for example, about 6 mg/kg once every 21 days, about 7.2 mg/kg once every 21 days, or about 8.4 mg/kg once every 21 days; more preferably the conjugate comprised in the kit is for intravenous administration; and/or
the additional cancer therapeutic agent is pyrotinib; preferably, pyrotinib is for administration in an amount ranging from about 300 mg to about 350 mg once daily for 28 days per treatment cycle, for example, about 300 mg once daily for 28 days per treatment cycle, about 310 mg once daily for 28 days per treatment cycle, about 320 mg once daily for 28 days per treatment cycle, about 330 mg once daily for 28 days per treatment cycle, about 340 mg once daily for 28 days per treatment cycle or about 350 mg once daily for 28 days per treatment cycle, preferably about 320 mg once daily for 28 days per treatment cycle; more preferably, pyrotinib is for oral administration.

In an embodiment, provided is also a kit, comprising the conjugate, the additional cancer therapeutic agent and instructions to administer the compounds to a patient in need; wherein the conjugate is administered at an effective amount of about 5 mg/kg to 10 mg/kg per treatment cycle, and the additional cancer therapeutic agent is administered at an effective amount of about 100 mg to 600 mg once daily per treatment cycle.

In an embodiment, wherein each treatment cycle of the conjugate administration is independently about 1 week, about 2 weeks, about 3 weeks, about 4 weeks, about 5 weeks, about 6 weeks, about 7 weeks, or a range between any two of these values (inclusive) or any value therein.

In an embodiment, wherein each treatment cycle of the additional cancer therapeutic agent administration is independently about 1 week, about 2 weeks, about 3 weeks, about 4 weeks, about 5 weeks, about 6 weeks, about 7 weeks, or a range between any two of these values (inclusive) or any value therein.

In some embodiments, the patient is treated for a plurality of (e.g., 2, 3, or 4) treatment cycles. In some embodiments, the patient is treated until conditions are alleviated and no treatment is required. In some embodiments, the patient is treated with a plurality of treatment cycles of the conjugate and the additional cancer therapeutic agent.

In some embodiments, the conjugate and the additional cancer therapeutic agent may have identical or different administration cycles. In some embodiments, the conjugate and the additional cancer therapeutic agent are each separate dosage units administered in combination. In some embodiments, the conjugate can be administered before, after, or simultaneously with the additional cancer therapeutic agent. In some embodiments, the conjugate and the additional cancer therapeutic agent simultaneously form combined dosage units administered in combination.

The ratio of the total amounts of the conjugate of formula (I-1), (I-2), (I-3), and/or (I-4) to one or more additional cancer therapeutic agents in the pharmaceutical combination of the present disclosure can be varied, e.g. in order to cope with the needs of a patient sub-population to be treated or the needs of a single patient which different needs can be due to the particular disease, age, sex, body weight, etc.

### Treatment Method and Use

In a further aspect, provided is a method for treating a cell proliferative disorder, comprising administering a subject in need of such treatment an effective amount of the pharmaceutical combination of the present disclosure or the kit or pharmaceutical composition of the present disclosure. In an embodiment, the one or more conjugates and the one or more additional cancer therapeutic agents are administered simultaneously, separately or sequentially.

In a further aspect, provided is use of the pharmaceutical combination of the present disclosure for the manufacture of a medicament for treatment of a cell proliferative disorder.

In a further aspect, provided is a method for treating a cell proliferative disorder, comprising administering a subject in need of such treatment an effective amount of the conjugate of formula (I-1), (I-2), (1-3) and/or (I-4), and one or more additional cancer therapeutic agents. In an alternative embodiment of the present disclosure, the conjugate of formula (I-1), (I-2), (I-3) and/or (I-4) and the one or more additional cancer therapeutic agents are administered simultaneously, sequentially or separately.

The conjugate of formula (I-1), (I-2), (1-3) and/or (I-4) has outstanding anti-cancer effect, and good safety (WO 2015165413A). Therefore, in a yet further aspect, provided is a method for enhancing the efficacy of a cancer therapeutic agent comprising applying the conjugate of formula (I-1), (I-2), (I-3) and/or (I-4) in combination with the cancer therapeutic agent.

In a yet further aspect, provided is a method for treating HER2-expressing cancer (HER-2 positive cancer), comprising administering a mixture of the conjugate of formula (i-1) and the conjugate of formula (i-2), and an additional cancer therapeutic agent; wherein the additional cancer therapeutic agent is selected from the group consisting of: tyrosine kinase inhibitors, taxanes, and pyrimidine antimetabolites.

In an embodiment, the tyrosine kinase inhibitors are selected from the group consisting of: tucatinib, neratinib and pyrotinib, preferably tucatinib or pyrotinib. In an embodiment, the taxanes are selected from the group consisting of: docetaxel and paclitaxel, preferably paclitaxel. In an embodiment, the pyrimidine antimetabolites are selected from the group consisting of: cytarabine, capecitabine, ancitabine, gemcitabine, enocitabine, carmofur, dideoxyuridine, doxifluridine and floxuridine, preferably capecitabine.

In an embodiment, the HER2-expressing cancer is HER2-overexpressing cancer.

In an embodiment, the HER2-overexpressing cancer is cancer given a score of 2+ for the expression of HER2 in an immunohistochemical method.

In an embodiment, the HER2-overexpressing cancer is cancer given a score of 3+ for the expression of HER2 in an immunohistochemical method.

In an embodiment, the cancer has resistance or refractoriness to an existing anticancer drug, wherein the existing anticancer drug comprises at least one selected from the group consisting of trastuzumab emtansine, trastuzumab, pertuzumab, lapatinib, irinotecan, cisplatin, carboplatin, oxaliplatin, fluorouracil, gemcitabine, capecitabine, paclitaxel, docetaxel, doxorubicin, epirubicin, cyclophosphamide, mitomycin C, a tegafur-gimeracil-oteracil combination drug, cetuximab, panitumumab, bevacizumab, ramucirumab, regorafenib, a trifluridine-tipiracil combination drug, gefitinib, erlotinib, afatinib, methotrexate, pyrotinib, palbociclib, and pemetrexed.

In an embodiment, the HER2-positive cancer is selected from the group consisting of: gastric or esophagogastric cancer, breast cancer, vaginal cancer, salivary cancer, endometrial cancer, bladder cancer, non-small cell lung cancer (NSCLC), cervical cancer, ovarian cancer, colorectal cancer and pancreatic adenocarcinoma.

In an embodiment, provided is a method for preventing, alleviating or treating a cell proliferative disorder, tumor or cancer, comprising administering a subject in need of an effective amount of the conjugate (or formulation) and the additional cancer therapeutic agent (or formulation); wherein the conjugate is administered at an effective amount of about 5 mg/kg to 10 mg/kg per treatment cycle, and the additional cancer therapeutic agent is administered at an effective amount of about 100 mg to 600 mg once daily per treatment cycle.

In an embodiment, wherein each treatment cycle of conjugate administration is independently about 1 week, about 2 weeks, about 3 weeks, about 4 weeks, about 5 weeks, about 6 weeks, about 7 weeks, or a range between any two of these values (inclusive) or any value therein. In an embodiment, the conjugate is administered at an effective amount of about 4 mg/kg to about 10 mg/kg once every 18 days to every 28 days; for example, about 4 mg/kg, about 5 mg/kg, about 6 mg/kg, about 7 mg/kg, about 7.2 mg/kg, about 8 mg/kg, about 8.4 mg/kg, about 9 mg/kg or about 10 mg/kg (or range between any two of these values) once every 18 days to every 28 days; about 4 mg/kg, about 5 mg/kg, about 6 mg/kg, about 7 mg/kg, about 8 mg/kg, about 9 mg/kg or about 10 mg/kg (or range between any two of these values) once every 18 days; or about 4 mg/kg, about 5 mg/kg, about 6 mg/kg, about 7 mg/kg, about 7.2 mg/kg, about 8 mg/kg, about 8.4 mg/kg, about 9 mg/kg or about 10 mg/kg (or range between any two of these values) once every 20 days; or about 4 mg/kg, about 5 mg/kg, about 6 mg/kg, about 7 mg/kg, about 7.2 mg/kg, about 8 mg/kg, about 8.4 mg/kg, about 9 mg/kg or about 10 mg/kg (or range between any two of these values) once every 21 days; or about 4 mg/kg, about 5 mg/kg, about 6 mg/kg, about 7 mg/kg, about 7.2 mg/kg, about 8 mg/kg, about 8.4 mg/kg, about 9 mg/kg or about 10 mg/kg (or range between any two of these values) once every 24 days; or about 4 mg/kg, about 5 mg/kg, about 6 mg/kg, about 7 mg/kg, about 7.2 mg/kg, about 8 mg/kg, about 8.4 mg/kg, about 9 mg/kg or about 10 mg/kg (or range between any two of these values) once every 28 days.

In an embodiment, the conjugate is administered at an effective amount of about 5 mg/kg to about 9 mg/kg once every 21 days. In an embodiment, the conjugate is administered at an effective amount of about 6 mg/kg to about 8.4 mg/kg once every 21 days, for example, about 6 mg/kg, about 7.2 mg/kg, or about 8.4 mg/kg once every 21 days. In a very special embodiment, the conjugate is administered at an effective amount of about 7.2 mg/kg once every 21 days.

In an embodiment, the conjugate is administered at an effective amount of about 200 mg to about 700 mg once every 18 days to every 28 days. In an embodiment, the conjugate is administered at an effective amount of about 200 mg to about 700 mg once every 18 days. In an embodiment, the conjugate is administered at an effective amount of about 200 mg to about 700 mg once every 21 days. In an embodiment, the conjugate is administered at an effective amount of about 200 mg to about 700 mg once every 24 days. In an embodiment, the conjugate is administered at an effective amount of about 200 mg to about 700 mg once every 28 days. In an embodiment, the conjugate is administered at an effective amount of about 200 mg, about 260 mg, about 300 mg, about 360 mg, about 400 mg, about 452 mg, about 460 mg, about 500 mg, about 504 mg, about 560 mg, about 600 mg, about 630 mg, about 660 mg, about 700 mg (or range between any two of these values) once every 18 days to every 28 days, for example once every 21 days. In an embodiment, the conjugate is administered at an effective amount of about 360 mg, about 452 mg or about 504 mg once every 21 days.

In an embodiment, the conjugate comprises structure of formula (i-1) and/or formula (i-2). In some embodiment, the conjugate consists of structure of formula (i-1) and formula (i-2).

In an embodiment, wherein each treatment cycle of the additional cancer therapeutic agent administration is independently about 1 week, about 2 weeks, about 3 weeks, about 4 weeks, about 5 weeks, about 6 weeks, about 7 weeks, or a range between any two of these values (inclusive) or any value therein. In an embodiment, the additional cancer therapeutic agent is administered at an effective amount of about 100 mg to about 600 mg once daily for 18 days to 30 days per treatment cycle; for example, about 100 mg to about 500 mg, or about 200 mg to about 400 mg, or about 260 mg to about 400 mg, or about 300 mg to about 350 mg once daily for 18 days per treatment cycle; about 100 mg to about 500 mg, or about 200 mg to about 400 mg, or about 260 mg to about 400 mg, or about 300 mg to about 350 mg once daily for 21 days per treatment cycle; about 100 mg to about 500 mg, or about 200 mg to about 400 mg, or about 260 mg to about 400 mg, or about 300 mg to about 350 mg once daily for 24 days per treatment cycle; about 100 mg to about 500 mg, or about 200 mg to about 400 mg, or about 260 mg to about 400 mg, or about 300 mg to about 350 mg once daily for 28 days per treatment cycle; about 100 mg to about 500 mg, or about 200 mg to about 400 mg, or about 260 mg to about 400 mg, or about 300 mg to about 350 mg once daily for 30 days per treatment cycle; preferably from about 300 mg to about 350 mg once daily for 18 days to 30 days per treatment cycle; for example, about 300 mg, about 310 mg, about 315 mg, about 320 mg, about 326 mg, about 330 mg, about 340 mg or about 350 mg once daily for 18 days to 30 days per treatment cycle. In an embodiment, the additional cancer therapeutic agent is administered at an effective amount of about 320 mg once daily for 28 days per treatment cycle.

In an embodiment, the additional cancer therapeutic agent is tyrosine kinase inhibitors, taxanes, or pyrimidine antimetabolites. In an embodiment, the additional cancer therapeutic agent is tucatinib, neratinib, pyrotinib, paclitaxel or capecitabine. In an embodiment, the cell proliferative disorder is HER2-positive breast cancer; preferably HER2-positive metastatic breast cancer; and/or the subject has received prior treatment(s) selected from the group consisting of: trastuzumab, pertuzumab, pyrotinib, capecitabine, palbociclib, letrozole and combination thereof.

In some embodiments, the patient is treated for a plurality of (e.g., 2, 3, 4, 5, 6, 7, 8, 9, 10, 11, 12, 13 or 14) treatment cycles. In some embodiments, the patient is treated until conditions are alleviated and no treatment is required. In some embodiments, the patient is treated with a plurality of treatment cycles of the conjugate and the additional cancer therapeutic agent.

In some embodiments, the conjugate and the additional cancer therapeutic agent may have identical or different administration cycles.

In some embodiments, the conjugate and the additional cancer therapeutic agent are each separate dosage units administered in combination. In some embodiments, the conjugate can be administered before, after, or simultaneously with the additional cancer therapeutic agent. In some embodiments, the conjugate and the additional cancer therapeutic agent simultaneously form combined dosage units administered in combination.

In a yet further aspect, provided is use of a conjugate of formula (I-1), (I-2), (I-3) and/or (I-4) for the manufacture of a medicament for prevention, alleviation or treatment of a cell proliferative disorder, wherein the medicament is for use in combination with one or more cancer therapeutic agents.

In an embodiment, the conjugate is a conjugate of formula (I-1) and/or (I-2). In another embodiment, the conjugate is a conjugate of formula (I-3) and/or (I-4). In an embodiment, the conjugate is a conjugate of formula (i-1) and/or formula (i-2). In an embodiment, the conjugate is mixture of formula (i-1) and formula (i-2).

In an embodiment, the cell proliferative disorder is a proliferative disorder associated with wild-type or mutant for HER2, or a proliferative disorder have amplification of HER2 (ErbB2) or have over expression of HER2 protein. In an embodiment, the cell proliferative disorder is a cancer. In an embodiment, the cancer is a HER2-positive cancer; wherein, preferably, the HER2 expression level is IHC 2+ or 3+, particularly 3+. In an embodiment, the cell proliferative disorder is a cancer associated with wild-type or mutant for HER2, or a proliferative disorder have amplification of HER2 (ErbB2) or have over expression of HER2 protein. In an embodiment, the cell proliferative disorder is characterized by increased proliferation and survival of HER2-positive tumor cells, especially human breast cancer or human gastric cancer cell. In some embodiments, the cancers are primary drug-resistant or secondary drug-resistant.

In an embodiment, the cell proliferative disorder is a cancerous tumor. In an embodiment, the cancer is selected from the group consisting of: gastric or esophagogastric cancer, breast cancer, vaginal cancer, salivary cancer, endometrial cancer, bladder cancer, non-small cell lung cancer (NSCLC), cervical cancer, ovarian cancer, colorectal cancer and pancreatic adenocarcinoma. In a preferred embodiment, the cancer is gastric or esophagogastric cancer, breast cancer, vaginal cancer, colorectal cancer, non-small cell lung cancer, and ovarian cancer. In a specific embodiment, the cancer is gastric or esophagogastric cancer, or breast cancer. In a very specific embodiment, the cancer is breast cancer. In an embodiment, the breast cancer is a mammary gland tumor.

In an embodiment, the cell proliferative disorder is HER2-positive breast cancer; preferably HER2-positive metastatic breast cancer.

In an embodiment, the pharmaceutical combination of the present disclosure is used as a first-line treatment. In an embodiment, the pharmaceutical combination of the present disclosure is used as a second- or later-line treatment. In an embodiment, the prior treatment(s) comprises/comprise antibody therapy, chemotherapy, and/or kinase inhibitor therapy; preferably antibody therapy and/or chemotherapy. In an embodiment, the prior treatment(s) is/are selected from the group consisting of: trastuzumab, pertuzumab, tucatinib, neratinib, pyrotinib, paclitaxel, capecitabine, palbociclib, letrozole and combination thereof. In an embodiment, the prior treatment(s) comprise antibody therapy, particularly treatment(s) using trastuzumab plus pertuzumab. In an embodiment, the prior treatment(s) comprise chemotherapy, particularly palbociclib therapy. In an embodiment, the prior treatment(s) comprise tyrosine kinase inhibitor therapy, particularly pyrotinib therapy. In an embodiment, the pharmaceutical combination of the present disclosure is used as neoadjuvant treatment. In an embodiment, the pharmaceutical combination of the present disclosure is used as adjuvant treatment. In an embodiment, the cell proliferative disorder is a cancer. In an embodiment, the cancer is breast cancer or gastric cancer, and the subject in need of such treatment has received prior treatment(s). In an embodiment, the subject in need of such treatment is resistant to one or more of the prior treatment(s). In an embodiment, the prior treatment(s) is/are selected from the group consisting of: trastuzumab, pertuzumab, tucatinib, neratinib, pyrotinib, paclitaxel, capecitabine, palbociclib, letrozole and combination thereof; preferably, the prior treatment(s) is/are selected from the group consisting of: trastuzumab, pertuzumab, pyrotinib, capecitabine, palbociclib, letrozole and combination thereof; more preferably, the prior treatment(s) is/are selected from the group consisting of: trastuzumab, pertuzumab, pyrotinib, palbociclib, and combination thereof. In an embodiment, the prior treatments include trastuzumab, pertuzumab, pyrotinib and capecitabine treatments. In another embodiment, the prior treatments include palbociclib and letrozole treatments.

In an embodiment, the treatment method and use of the present disclosure extends overall survival (OS). In an embodiment, the pharmaceutical combination of the present disclosure results in a synergistic effect, for example, on tumor inhibition. In an embodiment, the treatment method and use of the present disclosure does not result in an increase of side-effects relative to the corresponding monotherapies. In an embodiment, the treatment method and use of the present disclosure improves the overall response rate (ORR) or complete response (CR) or partial response (PR) relative to the corresponding monotherapies. In an embodiment, the treatment method and use of the present disclosure extends progression-free survival (PFS). In an embodiment, the treatment method and use of the present disclosure tumor improves the growth inhibition rate (TGI).

With respect to the treatment method and use of the present disclosure, the following embodiments regarding additional cancer therapeutic agent are encompassed and exemplified.
The additional cancer therapeutic agent is selected from the group consisting of: kinase inhibitors and chemotherapeutic agents. The cell proliferative disorder is, for example, a cancerous tumor.
The additional cancer therapeutic agent is selected from the group consisting of: tyrosine kinase inhibitors, taxanes, and pyrimidine antimetabolites. The cell proliferative disorder is, for example, gastric or esophagogastric cancer, or breast cancer.
The additional cancer therapeutic agent is selected from the group consisting of: HER family tyrosine kinase inhibitor. The cell proliferative disorder is, for example, gastric or esophagogastric cancer, or breast cancer.
The additional cancer therapeutic agent is selected from the group consisting of: tucatinib, neratinib, pyrotinib, paclitaxel and capecitabine. The cell proliferative disorder is, for example, breast cancer.
The additional cancer therapeutic agent is selected from the group consisting of: tucatinib and pyrotinib. The cell proliferative disorder is, for example, gastric or esophagogastric cancer, or breast cancer.
The additional cancer therapeutic agent is pyrotinib. The cell proliferative disorder is, for example, breast cancer. In an embodiment, the breast cancer is metastatic, recurrent, refractory or advanced. In an embodiment, the subject in need of treatment for breast cancer is resistant to antibody therapy, particularly trastuzumab plus pertuzumab therapy. Preferably, such resistance is acquired after trastuzumab plus pertuzumab therapy. In an embodiment, the subject in need of treatment for breast cancer is resistant to kinase inhibitor therapy, preferably CDK kinase inhibitor (e.g., CDK4/6 inhibitor) therapy, particularly palbociclib therapy. In an embodiment, the subject in need of treatment for breast cancer is resistant to combination of trastuzumab, pertuzumab, pyrotinib and capecitabine. In an embodiment, the subject in need of treatment for breast cancer is resistant to combination of palbociclib and letrozole.
The additional cancer therapeutic agent is pyrotinib or tucatinib. The cell proliferative disorder is, for example, gastric or esophagogastric cancer. In an embodiment, the subject in need of treatment for gastric cancer is resistant to combination of trastuzumab, pertuzumab, pyrotinib and capecitabine. In an embodiment, the subject in need of treatment for breast cancer is resistant to combination of palbociclib and letrozole.
The additional cancer therapeutic agent is tucatinib. The cell proliferative disorder is, for example, breast cancer. In an embodiment, the breast cancer is metastatic, recurrent, refractory or advanced. In an embodiment, the breast cancer is metastatic. In an embodiment, the subject in need of treatment for breast cancer is resistant to antibody therapy, particularly trastuzumab plus pertuzumab therapy. Preferably, such resistance is acquired after trastuzumab plus pertuzumab treatment. In an embodiment, the subject in need of treatment for breast cancer is resistant to kinase inhibitor therapy, preferably CDK kinase inhibitor (e.g., CDK4/6 inhibitor) therapy, particularly palbociclib therapy. In an embodiment, the subject in need of treatment for breast cancer is resistant to pyrotinib therapy. In an embodiment, the subject in need of treatment for breast cancer is resistant to combination of trastuzumab, pertuzumab, pyrotinib and capecitabine. In an embodiment, the subject in need of treatment for breast cancer is resistant to combination of palbociclib and letrozole.
The additional cancer therapeutic agent is tucatinib. The cell proliferative disorder is, for example, gastric or esophagogastric cancer.
The additional cancer therapeutic agent is selected from the group consisting of: taxanes. The cell proliferative disorder is, for example, breast cancer. In an embodiment, the taxane is paclitaxel.
The additional cancer therapeutic agent is selected from the group consisting of: pyrimidine antimetabolites. The cell proliferative disorder is, for example, breast cancer. In an embodiment, the pyrimidine antimetabolite is capecitabine.

### Beneficial Effects

The pharmaceutical combination of the present disclosure achieves at least one of the following technical effects:
(1) Good anti-cancer effect, especially in the aspects of improving overall response rate (ORR) or complete response (CR) or partial response (PR) or tumor growth inhibition rate (TGI).
(2) Good physicochemical properties (e.g., solubility, physical and/or chemical stability).
(3) Good pharmacokinetic properties (e.g., good stability in plasma, appropriate half-life and duration of action).
(4) Good safety (low toxicity on non-target normal cells or tissues, and/or fewer side effects, wider treatment window), etc.
(5) May be used for prevention, alleviation or treatment of a subject who is resistant to antibody therapy, particularly trastuzumab plus pertuzumab therapy.
(6) May be used for treatment of a subject who is resistant to kinase inhibitor therapy, preferably CDK kinase inhibitor therapy, particularly palbociclib therapy.

### Examples

### Preparation Example

In order to more clearly illustrate the objects and technical solutions, the present disclosure is further described below with reference to specific examples. It is to be understood that the examples are not intended to limit the scope of the disclosure. The specific experimental methods which were not mentioned in the following examples were carried out according to conventional experimental method.

### Abbreviations

CR, complete response; PR, partial response; SD, stable disease; PD, progressive disease; OS, overall survival; PFS, progression-free survival; IHC, immunohistochemistry; DAR, drug to antibody ratio; TGI, tumor growth inhibition rate.

### Instruments, Materials and Reagents

Unless otherwise stated, the instruments and reagents used in the examples are commercially available. The reagents can be used directly without further purification.

HER2 tests by IHC staining were conducted using IHC antibodies which are generally mutation-nonspecific.

GQ01 is mixture of the conjugate of formula (i-1) and the conjugate of formula (i-2), wherein the structures of formula (i-1) and formula (i-2) are as depicted above. The overall drug to antibody ratio (DAR) of GQ01 is about 1.8 by HIC-HPLC using Butyl column.

### Example 1 In Vivo Efficacy Evaluation of the Combination of GQ01 with TKIs in Human Breast Cancer PDX Model

Objective: To evaluate the in vivo anti-tumor efficacy of the combination of ADC drug GQ01 with TKIs in mice bearing human breast cancer HER2 IHC 3+ PDX model.

I. The tumor tissues of PDX model were extracted from the tumor-bearing mice and then transferred to culture medium, cut into small fragments, and inoculated subcutaneously at the right flank of SPF female NOD SCID mice aged 6~8 weeks.

II. The tumor diameter was measured with a caliper and the tumor volume was calculated according to the formula V=0.5 a×b² (wherein a is the long diameter of the tumor and b is the short diameter of the tumor). When the mean tumor volume was about 100-300 mm³, the mice were randomized into vehicle group, GQ01 5 mg/kg group, tucatinib 20 mg/kg group, neratinib 20 mg/kg group, pyrotinib 20 mg/kg group, GQ01+tucatinib combination group, GQ01+neratinib combination group, GQ01+pyrotinib combination group. Mice in the vehicle group were given the solvent of GQ01 and the solvent of pyrotinib with the same frequency and administration route. The tumor volume of mice in each group was measured twice a week. The experiment was ended on day 77, and the tumor growth inhibition rate (TGI) was calculated as follows: TGI (%)=[1−(the mean tumor volume of the treatment group on the end day−the mean tumor volume of the treatment group on the first day)/(the mean tumor volume of the vehicle group on the end day−the mean tumor volume of the vehicle group on the first day)]×100%.

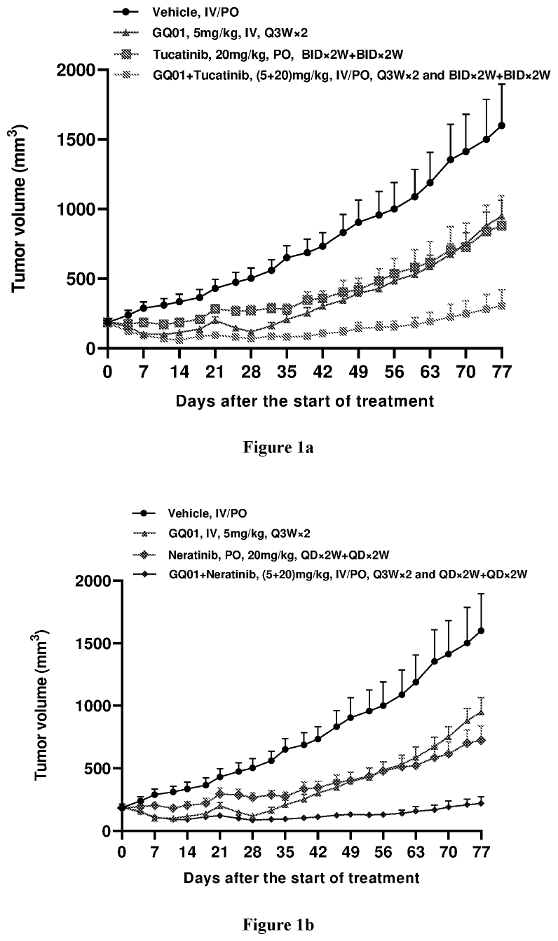

III. FIG. 1a showed the tumor volume change of tumor bearing NOD SCID female mice treated with: (1) vehicle, (2) GQ01 5 mg/kg (Q3W×2), (3) tucatinib 20 mg/kg (BID×2W+BID×2W), (4) GQ01 5 mg/kg (Q3W×2)+tucatinib 20 mg/kg (BID×2W+BID×2W). Table 1a showed that on the end day (day 77), the mean tumor volume in GQ01 5 mg/kg group, tucatinib 20 mg/kg group, GQ01 5 mg/kg+tucatinib 20 mg/kg group were 950 mm³, 879 mm³ and 307 mm³ respectively; TGI were 46.22%, 51.08% and 91.20% respectively.

These data show that, both GQ01 5 mg/kg and tucatinib 20 mg/kg as monotherapy can inhibit the growth of tumor, while the combination of GQ01 with tucatinib has better anti-tumor efficacy than the monotherapy.

TABLE la
Tumor growth inhibition of the combination of GQ01 with tucatinib
in human breast cancer PDX model calculated on tumor volume

| | | TGI^{b} |
|---|---|---|
| Group | Tumor volume (mm³) ^{a} | (%) |
| Vehicle | 1,600 ± 297 | — |
| GQ01 (5 mg/kg) | 950 ± 113 | 46.22% |
| Tucatinib (20 mg/kg) | 879 ± 216 | 51.08% |
| GQ01 + Tucatinib (5 + 20 mg/kg) | 307 ± 112 | 91.20% |

^{a.} Mean ± SEM; measured on the end day;
^{b}TGI (%) = [1-(T₇₇-T₀)/(V₇₇-V₀)] × 100%. To is the average tumor volume of the treatment group on the first day of administration, T₇₇ is the average tumor volume of the treatment group at day 77 after administration; V₀ is the average tumor volume of the vehicle group on the first day of administration, V₇₇ is the average tumor volume of the vehicle group at the day 77 after administration.

IV. FIG. 1b showed the tumor volume change of tumor bearing NOD SCID female mice treated with: (1) vehicle, (2) GQ01 5 mg/kg (Q3W×2), (3) neratinib 20 mg/kg (QD×2W+QD×2W), (4) GQ01 5 mg/kg (Q3W×2)+neratinib 20 mg/kg (QD×2W+QD×2W). Table 1b showed that on the end day (day 77), the mean tumor volume of GQ01 5 mg/kg group, neratinib 20 mg/kg group, GQ01 5 mg/kg+neratinib 20 mg/kg group were 950 mm³, 724 mm³ and 220 mm³ respectively; and TGI were 46.22%, 61.80% and 97.91% respectively.

These data show that both GQ01 5 mg/kg and neratinib 20 mg/kg as monotherapies can inhibit the growth of tumor. Compared with monotherapy group, the combination of GQ01 with neratinib shows superior tumor inhibition efficacy, which can inhibit the growth of tumor more effectively.

TABLE 1b
Tumor growth inhibition of the combination of GQ01 with neratinib
in human breast cancer PDX model calculated on tumor volume

| | | TGI^{b} |
|---|---|---|
| Group | Tumor volume (mm³) ^{a} | (%) |
| Vehicle | 1,600 ± 297 | — |
| GQ01 (5 mg/kg) | 950 ± 113 | 46.22% |
| Neratinib (20 mg/kg) | 724 ± 114 | 61.80% |
| GQ01 + Neratinib (5 + 20 mg/kg) | 220 ± 55 | 97.91% |

^{a.} Mean ± SEM; measured on the end day;
^{b}TGI (%) = [1-(T₇₇-T₀)/(V77-V₀)] × 100%. T₀ is the average tumor volume of the treatment group on the first day of administration, and T₇₇ is the average tumor volume of the treatment group at day77 after administration; V₀ is the average tumor volume of the vehicle group on the first day of administration, V₇₇ is the average tumor volume of the vehicle group at the day 77 after administration.

V. FIG. 1c showed the tumor volume change of tumor bearing NOD SCID female mice treated with: (1) vehicle, (2) GQ01 5 mg/kg (Q3W×2), (3) pyrotinib 20 mg/kg (QD×2W+QD×2W), (4) GQ01 5 mg/kg (Q3W×2)+pyrotinib 20 mg/kg (QD×2W+QD×2W). Table 1c showed that on the end day (day 77), the mean tumor volume of GQ01 5 mg/kg group, pyrotinib 20 mg/kg group, GQ01 5 mg/kg+pyrotinib 20 mg/kg group were 950 mm³, 1108 mm³ and 202 mm³ respectively; TGI were 46.22%, 34.93% and 98.74% respectively.

These data show that both GQ01 5 mg/kg and pyrotinib 20 mg/kg as monotherapies can inhibit the growth of tumor in a certain extent; the combination of GQ01 with pyrotinib can significantly increase antitumor efficacy than GQ01 or pyrotinib monotherapy.

TABLE 1c
Tumor growth inhibition of the combination of GQ01 with pyrotinib
in human breast cancer PDX model calculated on tumor volume

| | | TGI^{b} |
|---|---|---|
| Group | Tumor volume (mm³) ^{a} | (%) |
| Vehicle | 1,600 ± 297 | — |
| GQ01 (5 mg/kg) | 950 ± 113 | 46.22% |
| Pyrotinib (20 mg/kg) | 1108 ± 202 | 34.93% |
| GQ01 + Pyrotinib (5 + 20 mg/kg) | 202 ± 34 | 98.74% |

^{a.} Mean ± SEM; measured on the end day;
^{b}TGI (%) = [1-(T₇₇-T₀)/(V77-V₀)] × 100%. T₀ is the average tumor volume of the treatment group on the first day of administration, and T₇₇ is the average tumor volume of the treatment group at day 77 after administration; V₀ is the average tumor volume of the vehicle group on the first day of administration, V₇₇ is the average tumor volume of the vehicle group at the day 77 after administration.

The results show that in mice bearing the tumor, which is a HER2 positive (IHC 3+) human breast cancer PDX model, GQ01 or TKI monotherapy can inhibit the proliferation of tumor cells in varying efficacy. Furthermore, the antitumor efficacy can be dramatically enhanced by the combination of GQ01 with tucatinib or neratinib or pyrotinib.

### Example 2 In Vivo Efficacy Evaluation of the Combination of GQ01 with TKIs in Human Breast Cancer PDX Model

Objective: To evaluate the in vivo anti-tumor efficacy of the combination of ADC drug GQ01 with TKI in mice bearing breast cancer HER2 IHC 3+PDX model. The model was derived from a patient with Stage III invasive breast cancer who developed metastases to lung and bone after surgery. After treatment of palbociclib and letrozole, the disease was judged to be progressive disease (PD), and the model was identified as primary resistance to palbociclib.

I. The tumor tissues of PDX model were extracted from the tumor-bearing mice and were transferred to culture medium, cut into small fragments, and inoculated subcutaneously in the right flank of SPF female NCG mice aged 6~8 weeks.

II. 30 days after inoculation, the tumor diameter was measured with a caliper and the tumor volume was calculated according to the formula V=0.5 a×b² (wherein a is the long diameter of the tumor and b is the short diameter of the tumor). When the mean tumor volume was about 100-300 mm³, the mice were randomized into vehicle group, GQ01 5 mg/kg group, tucatinib 50 mg/kg group, pyrotinib 20 mg/kg group, GQ01+tucatinib combination group, GQ01+pyrotinib combination group. Each group included 6 mice. The day of first administration was defined as day 0. Mice in the vehicle group were given the solvent of GQ01 and the solvent of tucatinib with the same frequency and administration route. The tumor volume of mice was measured twice a week. The experiment was ended on day 49, and the tumor growth inhibition rate (TGI) was calculated as follows: TGI (%)−[1−(the mean tumor volume of the treatment group on the end day−the mean tumor volume of the treatment group on the first day)/(the mean tumor volume of the vehicle group on the end day−the mean tumor volume of the vehicle group on the first day)]×100%.

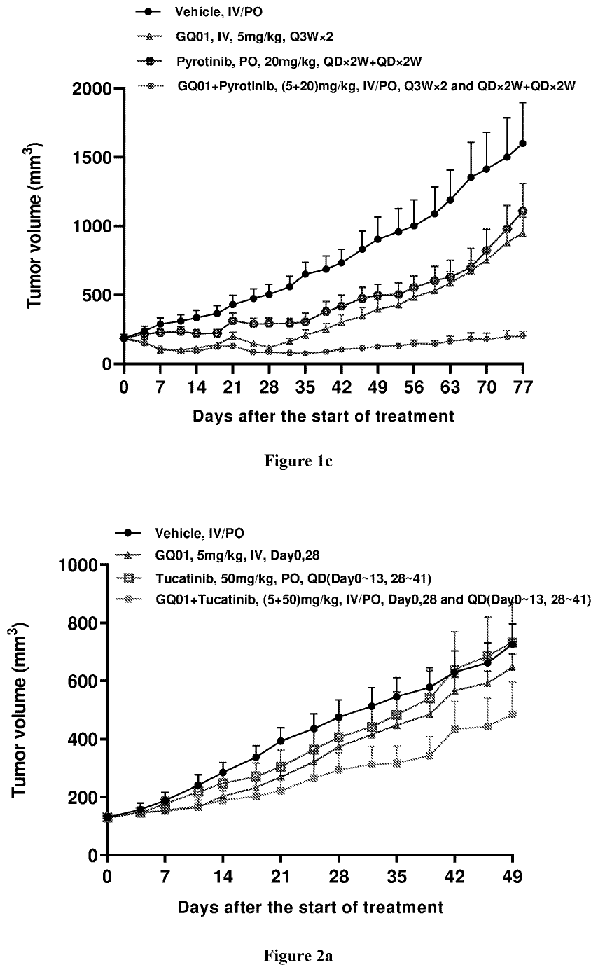

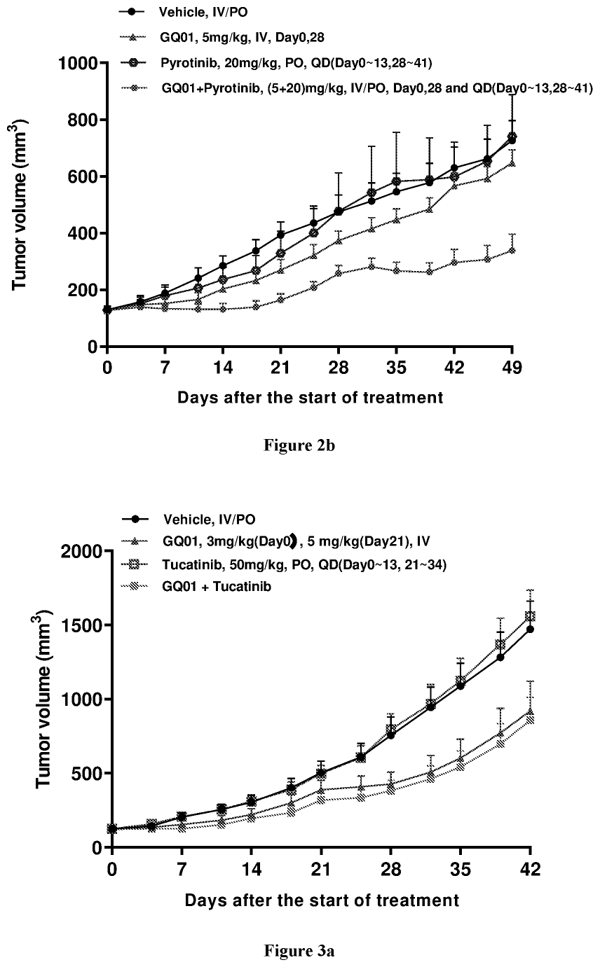

III. FIG. 2a showed the tumor volume change of tumor bearing NCG female mice treated with: (1) vehicle, (2) GQ01 5 mg/kg (on day 0 and day 28), (3) tucatinib 50 mg/kg (QD, day 0-13, 28-41), (4) GQ01 5 mg/kg (on day 0 and day 28)+tucatinib 50 mg/kg (QD, day 0-13, 28-41). Table 2a showed that on the end day (day 49), the mean tumor volume of GQ01 5 mg/kg group, tucatinib 50 mg/kg group, GQ01 5 mg/kg+tucatinib 50 mg/kg group were 648 mm³, 732 mm³ and 485 mm³ respectively; TGI were 12.92%, −1.09% and 40.16% respectively.

The results show that the combination of GQ01 with tucatinib can significantly inhibit the growth of this tumor than monotherapy.

TABLE 2a
Tumor growth inhibition of the combination of GQ01 with tucatinib
in human breast cancer PDX model calculated on tumor volume

| | | TGI^{b} |
|---|---|---|
| Group | Tumor volume (mm³) ^{a} | (%) |
| Vehicle | 726.03 ± 70.08 | — |
| GQ01 (5 mg/kg) | 647.57 ± 45.73 | 12.92% |
| Tucatinib (50 mg/kg) | 732.33 ± 137.71 | −1.09% |
| GQ01 + Tucatinib (5 + 50 mg/kg) | 485.1 ± 110.83 | 40.16% |

^{a.} Mean ± SEM; measured on the end day;
^{b}TGI (%) = [1-(T₄₉-T₀)/(V₄₉-V₀)] × 100%. T₀ is the mean tumor volume of the treatment group on the first day of administration, and T₄₉ is the mean tumor volume of the treatment group at day 49 after administration; V₀ is the average tumor volume of the vehicle group on the first day of administration, V₄₉ is the average tumor volume of the vehicle group at the day 49 after administration.

IV. FIG. 2b showed the tumor volume change of tumor bearing NCG female mice treated with: (1) vehicle, (2) GQ01 5 mg/kg (on day 0 and day 28), (3) pyrotinib 20 mg/kg (QD, day 0-13, 28-41), (4) GQ01 5 mg/kg (on day 0 and day 28)+pyrotinib 20 mg/kg (QD, day 0-13, 28-41). Table 2b showed that on the end day (day 49), the mean tumor volume of GQ01 5 mg/kg group, pyrotinib 20 mg/kg group, GQ01 5 mg/kg+pyrotinib 20 mg/kg group were 648 mm³, 741 mm³ and 338 mm³ respectively; TGI were 12.92%, −2.65% and 64.71% respectively.

From the result we find the combination of GQ01 with pyrotinib can inhibit the growth of tumor more effectively than GQ01 5 mg/kg or pyrotinib 20 mg/kg monotherapy.

TABLE 2b
Tumor growth inhibition of the combination of GQ01 with pyrotinib
in human breast cancer PDX model calculated on tumor volume

| | | TGI^{b} |
|---|---|---|
| Group | Tumor volume (mm³) ^{a} | (%) |
| Vehicle | 726.03 ± 70.08 | — |
| GQ01 (5 mg/kg) | 647.57 ± 45.73 | 12.92% |
| Pyrotinib (20 mg/kg) | 740.71 ± 146.76 | −2.65% |
| GQ01 + Pyrotinib (5 + 20 mg/kg) | 338.41 ± 57.35 | 64.71% |

^{a.} Mean ± SEM; measured on the end day;
^{b}TGI (%) = [1-(T₄₉-T₀)/(V₄₉-V₀)] × 100%. T₀ is the mean tumor volume of the treatment group on the first day of administration, and T₄₉ is the mean tumor volume of the treatment group at day 49 after administration; V₀ is the average tumor volume of the vehicle group on the first day of administration, V₄₉ is the average tumor volume of the vehicle group at the day 49 after administration.

The results show that in the metastatic and drug-resistant human breast cancer PDX model, TKIs as monotherapy cannot inhibit the growth of tumor, while the combination of GQ01 with tucatinib or pyrotinib can significantly increase the anti-tumor efficacy. The combination of ADC drug GQ01 and TKIs can benefit this type of patients.

### Example 3 In Vivo Efficacy Evaluation for the Combination of GQ01 with TKIs in Human Breast Cancer PDX Model

Objective: To evaluate the in vivo anti-tumor efficacy of the combination of ADC drug GQ01 and TKI in mice bearing human breast cancer HER2 IHC 3+PDX model. This model was derived from a patient with Stage III invasive breast cancer, who was assessed as acquired drug resistance to trastuzumab and pertuzumab and primary resistance to pyrotinib based on the history of treatments.

I. The tumor tissues of PDX model were extracted from the tumor-bearing mice and were transferred to culture medium, cut into small fragments, and then inoculated subcutaneously in the right flank of SPF female NCG mice aged 6~8 weeks.

II. 29 days after inoculation, the tumor diameter was measured with a caliper and the tumor volume was calculated according to the formula V=0.5 a×b2 (wherein a is the long diameter of the tumor and b is the short diameter of the tumor). When the mean tumor volume was about 100-300 mm³, the mice were randomized into vehicle group, GQ01 3, 5 mg/kg group, tucatinib 50 mg/kg group, pyrotinib 20 mg/kg group, GQ01+tucatinib combination group, GQ01+pyrotinib combination group. Each group included 6 mice. The day of first administration was defined as day 0. Mice in vehicle group were given the solvent of GQ01 and the solvent of tucatinib with same frequency and administration route. The tumor volume of mice in each group was measured twice a week. The experiment was ended on the day 42, and the tumor growth inhibition rate (TGI) was calculated as follows: TGI (%)=[1−(the mean tumor volume of the treatment group on the end day−the mean tumor volume of the treatment group on the first day)/(the mean tumor volume of the vehicle group on the end day−the mean tumor volume of the vehicle group on the first day)]×100%.

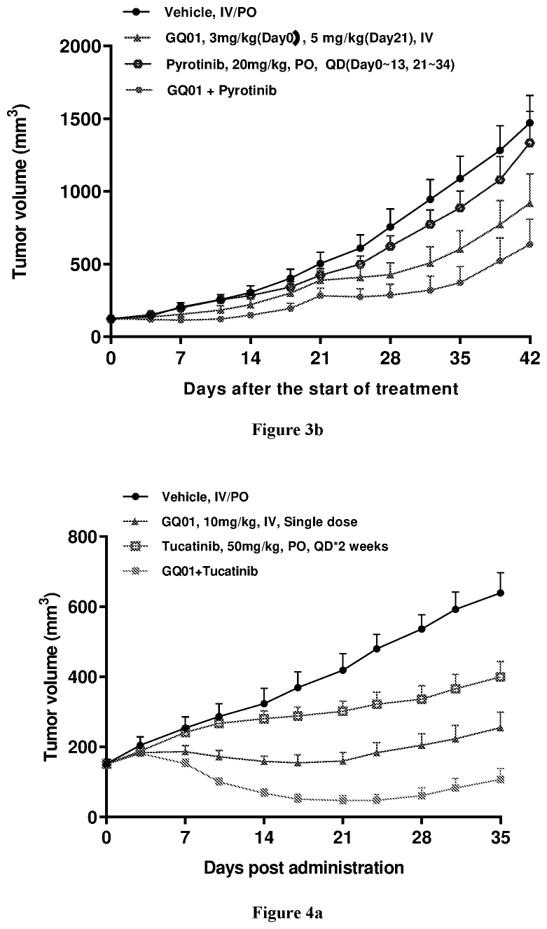

III. FIG. 3a showed the tumor volume change of tumor bearing mice treated with: (1) vehicle, (2) GQ01 3/5 mg/kg (3 mg/kg on day 0 and 5 mg/kg on day 21), (3) tucatinib 50 mg/kg (QD, day 0-13, 21-34), (4) GQ01 3/5 mg/kg (3 mg/kg on day 0 and 5 mg/kg on day 21)+tucatinib 50 mg/kg (QD, day 0-13, 21-34). Table 3a showed that on the end day (day 42), the mean tumor volume of GQ01 3/5 mg/kg group, tucatinib 50 mg/kg group, GQ01 3/5 mg/kg+tucatinib 50 mg/kg group were 920 mm³, 1558 mm³ and 857 mm³ respectively; TGI were 40.97%, −6.46% and 45.44% respectively.

According to the change of tumor volume and TGI, GQ01 monotherapy can inhibit the growth of tumor in a certain extent, the model doesn't respond to tucatinib treatment, and combination of GQ01 with tucatinib has better anti-tumor efficacy.

TABLE 3a
Tumor growth inhibition of the combination of GQ01 with tucatinib
in human breast cancer PDX model calculated on tumor volume

| | | TGI^{b} |
|---|---|---|
| Group | Tumor volume (mm³) ^{a} | (%) |
| Vehicle | 1470.78 ± 190.24 | — |
| GQ01 (3/5 mg/kg) | 919.62 ± 201.22 | 40.97% |
| Tucatinib (50 mg/kg) | 1557.77 ± 178.71 | −6.46% |
| GQ01 + Tucatinib (3/5 + 50 mg/kg) | 857.22 ± 154.79 | 45.44% |

^{a.} Mean ± SEM; measured on the end day;
^{b}TGI (%) = [1-(T₄₂-T₀)/(V₄₂-V₀)] × 100%. T₀ is the mean tumor volume of the treatment group on the first day of administration, and T₄₂ is the mean tumor volume of the treatment group at day 42 after administration; V₀ is the average tumor volume of the vehicle group on the first day of administration, V₄₂ is the average tumor volume of the vehicle group at the day 42 after administration.

IV. FIG. 3b showed the tumor volume change of tumor bearing mice treated with: (1) vehicle, (2) GQ01 3/5 mg/kg (3 mg/kg on day 0 and 5 mg/kg on day 21), (3) pyrotinib 20 mg/kg (QD, day 0-13, 21-34), (4) GQ01 3/5 mg/kg (3 mg/kg on day 0 and 5 mg/kg on day 21)+pyrotinib 20 mg/kg (QD, day 0-13, 21-34). Table 3b showed that on the end day (day 42), the mean tumor volume of GQ01 3/5 mg/kg group, pyrotinib 20 mg/kg group, GQ01 3/5 mg/kg+pyrotinib 20 mg/kg group were 920 mm³, 1333 mm³ and 634 mm³ respectively; TGI were 40.97%, 10.08% and 62.09% respectively.

According to the change of tumor volume and TGI, GQ01 monotherapy shows efficacy in tumor inhibition. On this pyrotinib resistant breast cancer model, combination of GQ01 with pyrotinib can significantly inhibit the growth of tumor, showing better anti-tumor efficacy than the monotherapy.

TABLE 3b
Tumor growth inhibition of the combination of GQ01 with pyrotinib
in human breast cancer PDX model calculated on tumor volume

| | | TGI^{b} |
|---|---|---|
| Group | Tumor volume (mm³) ^{a} | (%) |
| Vehicle | 1470.78 ± 190.24 | — |
| GQ01 (3/5 mg/kg) | 919.62 ± 201.22 | 40.97% |
| Pyrotinib (20 mg/kg) | 1333.21 ± 217.85 | 10.08% |
| GQ01 + Pyrotinib ( 3/5 + 20 mg/kg) | 634.16 ± 174.47 | 62.09% |

^{a.} Mean ± SEM; measured on the end day;
^{b}TGI (%) = [1-(T₄₂-T₀)/ (V₄₂-V₀)] × 100%. T₀ is the mean tumor volume of the treatment group on the first day of administration, and T₄₂ is the mean tumor volume of the treatment group at day 42 after administration; V₀ is the average tumor volume of the vehicle group on the first day of administration, V₄₂ is the average tumor volume of the vehicle group at the day 42 after administration.

The results show that in TKI-resistant HER2-positive (IHC 3+) breast cancer model, GQ01 monotherapy has a trend of increased efficacy compared with the vehicle, and the combination of GQ01 with TKIs presents stronger tumor inhibition.

### Example 4 In Vivo Efficacy Evaluation for the Combination of GQ01 with TKIs in Human Gastric Cancer PDX Model

Objective: To evaluate the in vivo anti-tumor efficacy of the combination of ADC drug GQ01 with TKI in mice bearing gastric cancer HER2 IHC 3+ PDX model

I. The tumor tissues of PDX model were extracted from the tumor-bearing mice and were transferred to RPMI-1640 culture medium, cut into small fragments, and then inoculated subcutaneously in the right flank of SPF female Nu/Nu mice aged 6~8 weeks.

II. 14 days after inoculation, the tumor diameter was measured with a caliper and the tumor volume was calculated according to the formula V=0.5 a×b2 (wherein a is the long diameter of the tumor and b is the short diameter of the tumor). When the mean tumor volume was about 100-300 mm³, the mice were randomized into vehicle group, GQ01 10 mg/kg group, tucatinib 50 mg/kg group, pyrotinib 20 mg/kg group, GQ01+tucatinib combination group, GQ01+pyrotinib combination group. Each group included 6 mice. The day of first administration was defined as day 0. Mice in the vehicle group were given both the solvent of GQ01 and the solvent of pyrotinib with the same frequency and administration route. The tumor volume of mice in each group was measured twice a week. The experiment was ended on day 35, and the tumor growth inhibition rate (TGI) was calculated as follows: TGI (%)=[1−(the mean tumor volume of the treatment group on the end day−the mean tumor volume of the treatment group on the first day)/(the mean tumor volume of the vehicle group on the end day the mean tumor volume of the vehicle group on the first day)]×100%.

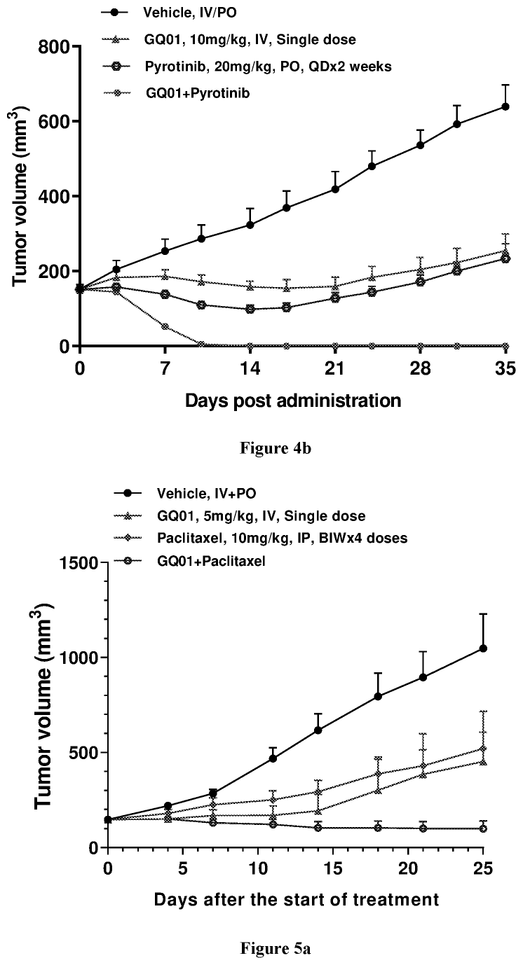

III. FIG. 4a showed the tumor volume change of tumor bearing mice treated with: (1) vehicle, (2) GQ01 10 mg/kg (single dose), (3) tucatinib 50 mg/kg (QD, 2 weeks), (4) GQ01 10 mg/kg (single dose)+tucatinib 50 mg/kg (QD, 2 weeks). Table 4a showed that on the end day (day 35), the mean tumor volume of GQ01 10 mg/kg group, tucatinib 50 mg/kg group, GQ01 10 mg/kg+tucatinib 50 mg/kg group were 255 mm³, 400 mm³ and 107 mm³ respectively; TGI were 78.83%, 48.94% and 108.94% respectively.

Tumor volume change and TGI rate indicate that both GQ01 10 mg/kg and tucatinib 50 mg/kg as monotherapy can inhibit the growth of tumor in a certain extent. The combination of GQ01 with tucatinib is significantly superior than GQ01 or tucatinib monotherapy, and causes 1/6 PR and even 1/6 CR.

TABLE 4a
Tumor growth inhibition of the combination of GQ01 with tucatinib
in human gastric cancer PDX model calculated on tumor volume

| | | TGI^{b} |
|---|---|---|
| Group | Tumor volume (mm³) ^{a} | (%) |
| Vehicle | 639.25 ± 58.21 | — |
| GQ01 (10 mg/kg) | 255.10 ± 44.54 | 78.83% |
| Tucatinib (50 mg/kg) | 399.97 ± 43.87 | 48.94% |
| GQ01 + Tucatinib (10 + 50 mg/kg) | 107.19 ± 31.45 | 108.94% |

^{a.} Mean ± SEM; measured on the end day;
^{b}TGI (%) = [1-(T₃₅-T₀)/(V₃₅-V₀)] × 100%. T₀ is the mean tumor volume of the treatment group on the first day of administration, and T₃₅ is the mean tumor volume of the treatment group at day 35 after administration; V₀ is the average tumor volume of the vehicle group on the first day of administration, V₃₅ is the average tumor volume of the vehicle group at the day 35 after administration.

The response status were calculated based on the diameters or disappearance of target lesions. The results were provided in the following table:

| | Number | | | | |
|---|---|---|---|---|---|
| | of animals | | | | |
| | at the | | | | |
| Group | start/end | CR | PR | SD | PD |
| Vehicle | 6/6 | — | — | — | 100.00% |
| GQ01 | 6/6 | — | — | 33.33% | 66.67% |
| Tucatinib | 6/6 | — | — | — | 100% |
| GQ01 + | 6/6 | 16.67% | 16.67% | 50.00% | 16.67% |
| Tucatinib | | | | | |

IV. FIG. 4b showed the tumor volume change of tumor bearing mice treated with: (1) vehicle, (2) GQ01 10 mg/kg (single dose), (3) pyrotinib 20 mg/kg (QD, 2 weeks), (4) GQ01 10 mg/kg (single dose)+pyrotinib 20 mg/kg (QD, 2 weeks). Table 4b showed that on the end day (day 35), the mean tumor volume of GQ01 10 mg/kg group, pyrotinib 20 mg/kg group, GQ01 10 mg/kg+pyrotinib 20 mg/kg group were 255 mm³, 234 mm³ and 0 mm³ respectively; TGI were 78.83%, 83.09% and 131.17% respectively.

The results show that GQ01 or pyrotinib monotherapy can inhibit the growth of tumor, and the combination of GQ01 with pyrotinib has stronger anti-tumor activity than monotherapy, and leads to complete tumor regression in all mice.

TABLE 4b
Tumor growth inhibition of the combination of GQ01 with pyrotinib
in human gastric cancer PDX model calculated on tumor volume

| | | TGI^{b} |
|---|---|---|
| Group | Tumor volume (mm³) ^{a} | (%) |
| Vehicle | 639.25 ± 58.21 | — |
| GQ01 (10 mg/kg) | 255.10 ± 44.54 | 78.83% |
| Pyrotinib (20 mg/kg) | 233.72 ± 39.7 | 83.09% |
| GQ01 + Pyrotinib (10 + 20 mg/kg) | 0 ± 0 | 131.17% |

^{a.} Mean ± SEM; measured on the end day;
^{b}TGI (%) = [1-(T₃₅-T₀)/(V₃₅-V₀)] × 100%. T₀ is the mean tumor volume of the treatment group on the first day of administration, and T₃₅ is the mean tumor volume of the treatment group at day 35 after administration; V₀ is the average tumor volume of the vehicle group on the first day of administration, V₃₅ is the average tumor volume of the vehicle group at the day 35 after administration.

The response status were calculated based on the diameters or disappearance of target lesions. The results were provided in the following table:

| | Number of animals | | | | |
|---|---|---|---|---|---|
| Group | at the start/end | CR | PR | SD | PD |
| Vehicle | 6/6 | — | — | — | 100.00% |
| GQ01 | 6/6 | — | — | 33.33% | 66.67% |
| Pyrotinib | 6/6 | — | — | 16.67% | 83.33% |
| GQ01 + Pyrotinib | 6/6 | 100% | | | — |

The results show that in HER2-positive (IHC 3+) gastric cancer model, GQ01 and TKI as monotherapy can inhibit the proliferation of tumor cells, and the combination of GQ01 with TKIs show significantly enhanced anti-tumor activities, results in increased overall response rate (ORR) or complete response (CR) or partial response (PR) or tumor growth inhibition rate (TGI).

### Example 5 In Vivo Efficacy Evaluation for the Combination of GQ01 with Chemotherapies in Human Breast Cancer CDX Model

Objective: To evaluate the in vivo anti-tumor efficacy of the combination of ADC drug GQ01 with chemotherapies in mice bearing breast cancer HER2-high CDX model.

I. Breast cancer tumor cells were passaged in vivo from BT-474 cells (ATCC, Lot #HTB-20). The cells in exponential growth stage were collected and counted for tumor inoculation. 0.2 mL of matrix gel buffer (PBS: Matrigel=1:1) was used to subcutaneously inject 2×10⁶ cells into the right flank of SPF female BALB/c nude mice aged 6-8 weeks. Four days before cell inoculation, BALB/c nude mice had been subcutaneously inoculated with estrogen tablet.

II. 10 days after inoculation, the tumor diameter was measured with a caliper and the tumor volume was calculated according to the formula V=0.5 a×b2 (wherein a is the long diameter of the tumor and b is the short diameter of the tumor). When the mean tumor volume was about 100-300 mm³, the mice were randomized into vehicle group, GQ01 5 mg/kg group, paclitaxel 10 mg/kg group, capecitabine 300 mg/kg group, GQ01+paclitaxel combination group, and GQ01+capecitabine combination group. Each group included 6 mice. The day of first administration was defined as day 0. Mice in the vehicle group were given the solvent of GQ01 and the solvent of capecitabine with the same frequency and administration route. The tumor volume of mice in each group was measured twice a week. The experiment was ended on day 25, and the tumor growth inhibition rate (TGI) was calculated as follows: TGI (%)=[1−(the mean tumor volume of the treatment group on the end day−the mean tumor volume of the treatment group on the first day)/(the mean tumor volume of the vehicle group on the end day−the mean tumor volume of the vehicle group on the first day)]×100%.

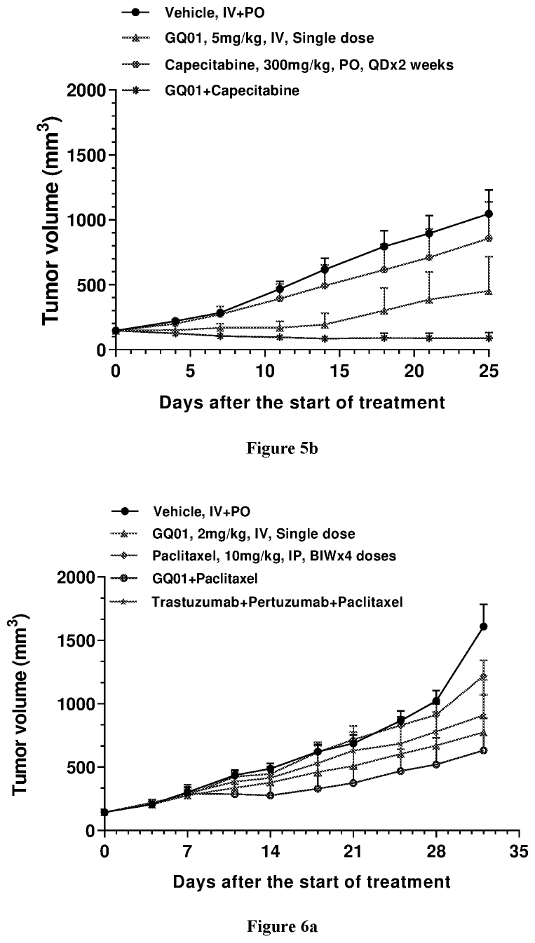

III. FIG. 5a showed the tumor volume change of tumor bearing mice treated with: (1) vehicle, (2) GQ01 5 mg/kg (single dose), (3) paclitaxel 10 mg/kg (BIW, 4 weeks), (4) GQ01 5 mg/kg (single dose)+paclitaxel 10 mg/kg (BIW, 4 weeks). Table 5a showed after 25 days of administration, the mean tumor volumes of GQ01 5 mg/kg group, paclitaxel 10 mg/kg group, GQ01 5 mg/kg+paclitaxel 10 mg/kg group were 452 mm³, 520 mm³ and 99 mm³ respectively; TGI were 66.04%, 58.54% and 105.27% respectively.

These data show that all monotherapy groups can inhibit the growth of tumor, and the combination of GQ01 with paclitaxel significantly decreases the volume of tumor, indicates excellent anti-tumor efficacy compared with paclitaxel or GQ01 monotherapy.

TABLE 5a
Tumor growth inhibition of the combination of GQ01 with
chemotherapies in breast cancer CDX model calculated on tumor volume

| | | TGI^{b} |
|---|---|---|
| Group | Tumor volume (mm³) ^{a} | (%) |
| Vehicle | 1047 ± 181 | — |
| GQ01 5 mg/kg | 452 ± 264 | 66.04% |
| Paclitaxel 10 mg/kg | 520 ± 87 | 58.54% |
| GQ01 + Paclitaxel (5 + 10 mg/kg) | 99 ± 41 | 105.27% |

^{a.} Mean ± SEM; measured on the end day;
^{b}TGI (%) = [1-(T₂₅-T₀)/(V₂₅-V₀)] × 100%. T₀ is the mean tumor volume of the treatment group on the first day of administration, and T₂₅ is the mean tumor volume of the treatment group at day 25 after administration; V₀ is the average tumor volume of the vehicle group on the first day of administration, V₂₅ is the average tumor volume of the vehicle group at the day 25 after administration.

IV. FIG. 5b showed the tumor volume change of tumor bearing mice treated with: (1) vehicle, (2) GQ01 5 mg/kg (single dose), (3) capecitabine, 300 mg/kg (QD, 2 weeks), (4) GQ01 5 mg/kg (single dose)+capecitabine, 300 mg/kg (QD, 2 weeks). Table 5b showed that on the end day (day 25), the mean tumor volume of GQ01 5 mg/kg group, capecitabine 300 mg/kg group, GQ01 5 mg/kg+capecitabine 300 mg/kg group were 452 mm³, 858 mm³ and 89 mm³ respectively; TGI were 66.04%, 21.01% and 106.41% respectively.

The results show that GQ01 monotherapy can inhibit the growth of tumor, while the combination of GQ01 with capecitabine has superior anti-tumor activity than capecitabine or GQ01 monotherapy.

TABLE 5b
Tumor growth inhibition of the combination of GQ01 with chemotherapies
in breast cancer CDX model calculated on tumor volume

| | | TGI^{b} |
|---|---|---|
| Group | Tumor volume (mm³) ^{a} | (%) |
| Vehicle | 1047 ± 181 | — |
| GQ01 5 mg/kg | 452 ± 264 | 66.04% |
| Capecitabine, 300 mg/kg | 858 ± 280 | 21.01% |
| GQ01 + Capecitabine (5 + 300 mg/kg) | 89 ± 43 | 106.41% |

^{a.} Mean ± SEM; measured on the end day;
^{b}TGI (%) = [1-(T₂₅-T₀)/(V₂₅-V₀)] × 100%. T₀ is the mean tumor volume of the treatment group on the first day of administration, and T₂₅ is the mean tumor volume of the treatment group at day 25 after administration; V₀ is the average tumor volume of the vehicle group on the first day of administration, V₂₅ is the average tumor volume of the vehicle group at the day 25 after administration.

The results show that in HER2-positive (IHC 3+) breast cancer CDX model, GQ01 and chemotherapies as respective monotherapies can inhibit the proliferation of tumor cells, and the combinations of GQ01 with chemotherapies show significantly increased anti-tumor activities compared with monotherapies.

### Example 6 In Vivo Efficacy Evaluation for the Combination of GQ01 with Chemotherapies in Human Breast Cancer CDX Model

Objective: To evaluate the in vivo anti-tumor efficacy of the combination of ADC drug GQ01 with chemotherapies in mice bearing breast cancer HER2-high CDX model.

I. HCC1954 cells in exponential growth stage were collected and counted for tumor inoculation. 0.2 mL of matrix gel buffer (PBS: Matrigel=1:1) was used to subcutaneously inject 1×10⁶ cells into the right flank of SPF female BALB/c nude mice aged 6-8 weeks.

II. 9 days after inoculation, the tumor diameter was measured with a caliper and the tumor volume was calculated according to the formula V=0.5 a×b2 (wherein a is the long diameter of the tumor and b is the short diameter of the tumor). When the mean tumor volume was about 100-300 mm³, the mice were randomized into vehicle group, GQ01 2 mg/kg group, paclitaxel 10 mg/kg group, capecitabine 300 mg/kg group, GQ01+paclitaxel combination group, GQ01+capecitabine combination group, and trastuzumab 5 mg/kg+pertuzumab 5 mg/kg+paclitaxel 10 mg/kg group. Each group included 6 mice. The day of first administration was defined as day 0. Mice in the vehicle group were given the solvent of GQ01 and the solvent of capecitabine with the same frequency and administration route. The tumor volume of mice in each group was measured twice a week. The experiment was ended on day 32, and the tumor growth inhibition rate (TGI) was calculated as follows: TGI (%)=[1−(the mean tumor volume of the treatment group on the end day−the mean tumor volume of the treatment group on the first day)/(the mean tumor volume of the vehicle group on the end day−the mean tumor volume of the vehicle group on the first day)]×100%.

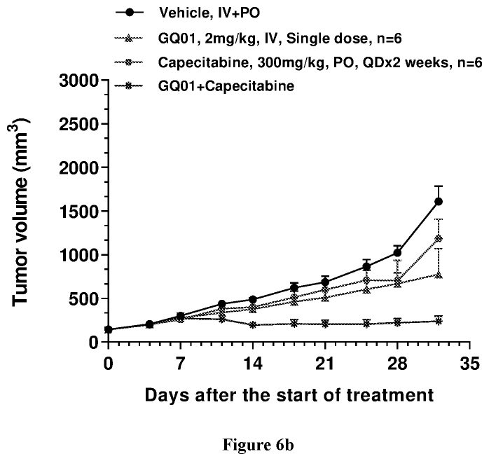

III. FIG. 6a showed the tumor volume change of tumor bearing BALB/c nude mice treated with: (1) vehicle, (2) GQ01 2 mg/kg (single dose), (3) paclitaxel 10 mg/kg (BIW, 4 doses), (4) GQ01 2 mg/kg (single dose)+paclitaxel 10 mg/kg (BIW, 4 doses), (5) trastuzumab 5 mg/kg (single dose)+pertuzumab 5 mg/kg (single dose)+paclitaxel 10 mg/kg (BIW, 4 doses). Table 6a showed that on the end day (day 32), the mean tumor volumes of GQ01 2 mg/kg group, paclitaxel 10 mg/kg group, GQ01 2 mg/kg+paclitaxel 10 mg/kg group and trastuzumab 5 mg/kg+pertuzumab 5 mg/kg+paclitaxel 10 mg/kg were 774 mm³, 1219 mm³, 632 mm³ and 909 mm³ respectively; TGI were 56.93%, 26.60%, 66.62% and 47.73% respectively.

The results show that GQ01 2 mg/kg as monotherapy can inhibit the growth of tumor cells. The combination of GQ01 with paclitaxel significantly inhibit the tumor growth, and the effect is better than the treatment of combination of trastuzumab, pertuzumab and paclitaxel.

TABLE 6a
Tumor growth inhibition of the combination of GQ01 with chemotherapies
in breast cancer CDX model calculated on tumor volume

| | Tumor volume | TGI^{b} |
|---|---|---|
| Group | (mm³) ^{a} | (%) |
| Vehicle | 1,609 ± 174 | — |
| GQ01 2 mg/kg | 774 ± 294 | 56.93% |
| Paclitaxel 10 mg/kg | 1,219 ± 124 | 26.60% |
| GQ01 + Paclitaxel, (2 + 10 mg/kg) | 632 ± 254 | 66.62% |
| Trastuzumab + Pertuzumab + Paclitaxel, | 909 ± 278 | 47.73% |
| (5 + 5 + 10 mg/kg) | | |

^{a.} Mean ± SEM; measured on the end day;
^{b}TGI (%) = [1-(T₃₂-T₀)/(V₃₂-V₀)] × 100%. T₀ is the mean tumor volume of the treatment group on the first day of administration, and T₃₂ is the mean tumor volume of the treatment group at day 32 after administration; V₀ is the average tumor volume of the vehicle group on the first day of administration, V₃₂ is the average tumor volume of the vehicle group at the day 32 after administration.

IV. FIG. 6b showed the tumor volume change of tumor bearing BALB/c female mice treated with: (1) vehicle, (2) GQ01 2 mg/kg (single dose), (3) capecitabine, 300 mg/kg (QD, 2 weeks), (4) GQ01 2 mg/kg (single dose)+capecitabine 300 mg/kg (QD, 2 weeks). Table 6b showed that on the end day (day 32), the mean tumor volume of GQ01 2 mg/kg group, capecitabine 300 mg/kg group, GQ01 2 mg/kg+capecitabine 300 mg/kg group were 774 mm³, 1183 mm³ and 238 mm³ respectively; TGI were 56.93%, 29.05% and 93.50% respectively.

The results show that GQ01 has anti-tumor efficacy as monotherapy, the combination of GQ01 with capecitabine significantly increase tumor inhibition ability than capecitabine or GQ01 monotherapy.

TABLE 6b
Tumor growth inhibition of the combination of GQ01 with chemotherapies
in breast cancer CDX model calculated on tumor volume

| | | TGI^{b} |
|---|---|---|
| Group | Tumor volume (mm³) ^{a} | (%) |
| Vehicle | 1,609 ± 174 | — |
| GQ01 5 mg/kg | 774 ± 294 | 56.93% |
| Capecitabine 300 mg/kg | 1,183 ± 222 | 29.05% |
| GQ01 + Capecitabine | 238 ± 57 | 93.50% |
| (5 + 300 mg/kg) | | |

^{a.} Mean ± SEM; measured on the end day;
^{b}TGI (%) = [1-(T₂₅-T₀)/(V₂₅-V₀)] × 100%. T₀ is the mean tumor volume of the treatment group on the first day of administration, and T₂₅ is the mean tumor volume of the treatment group at day 25 after administration; V₀ is the average tumor volume of the vehicle group on the first day of administration, V₂₅ is the average tumor volume of the vehicle group at the day 25 after administration.

The results show that in HER2-positive (IHC 3+) breast cancer CDX model, GQ01 and chemotherapies as respective monotherapies can inhibit the proliferation of tumor cells, and the combinations of GQ01 with chemotherapies have significantly increased anti-tumor efficacy compared with monotherapies.

### Example 7 Safety and Preliminary Efficacy of GQ01 Combined with Pyrotinib in Clinical Trial

### Objective:

A clinical phase study was designed to investigate the safety and preliminary efficacy of the combination of GQ01 and pyrotinib in pretreated patients with HER2-positive metastatic breast cancer.

### Methods

In the dose-escalation phase, the starting dose of GQ01 was 6.0 mg/kg intravenously once every 21 days (Q3W) and pyrotinib was 320 mg orally once daily (QD) in 28-day cycles. The combination dose finding of GQ01 and pyrotinib followed the “3+3” principle, with a subsequent dose escalation or de-escalation based on the incidence of specified dose-limiting toxicities (DLTs) in the initial dose group. DLTs were observed for 21 days. The dose of GQ01 was 6.0 mg/kg, 7.2 mg/kg and 8.4 mg/kg, respectively. Maximal tolerable dose (MTD) was not reached up to 8.4 mg/kg, the highest dose tested.

### Results:

A total of 15 HER2-positive metastatic breast cancer patients received GQ01 and pyrotinib treatment. All patients (15/15) had failed on previous trastuzumab (or its biosimilars) and chemotherapy. 33.3% of the patients (5/15) had been treated with pertuzumab. At the date of completion of the dose escalation, no DLT has been reported, and the Maximal tolerable dose (MTD) was not reached. 73.3% patients (13/15) experienced grade 3-4 treatment-related adverse event(s) (TRAE), including diarrhea (66.7%), anemia (13.3%), alanine aminotransferase increase (6.7%), thrombocytopenia (6.7%), and urticaria (6.7%).

At the date of completion of the dose escalation, nine patients were still on study treatment, with the longest treatment duration of 348 days. The objective response rate (ORR) was 66.7% (10/15) in all patients enrolled, and 71.4% (10/14) in patients who had received tumor evaluation. Analysis showed that the ORR was 75% (3/4), 100% (5/5), 33.3% (2/6) at 6.0 mg/kg, 7.2 mg/kg and 8.4 mg/kg dose level, respectively.

Ten days after the completion of the dose escalation, nine patients were still on study treatment, with the longest treatment duration of 358 days. Analysis showed that the objective response rate (ORR) was 73.3% (11/15) in all patients enrolled. ORR was 75% (3/4), 100% (5/5), 50% (3/6) at 6.0 mg/kg, 7.2 mg/kg and 8.4 mg/kg dose level, respectively.

### CONCLUSION

GQ01 in combination with pyrotinib had acceptable toxicity and showed preliminary antitumor activity among pretreated patients with HER2-positive metastatic breast cancer.

Sequence Listing
SEQ ID NO: 1: Trastuzumab LCDR1
RASQDVNTAVA
SEQ ID NO: 2: Trastuzumab LCDR2
SASFLYS
SEQ ID NO: 3: Trastuzumab LCDR3
QQHYTTPPT
SEQ ID NO: 4: Trastuzumab HCDR1
DTYIH
SEQ ID NO: 5: Trastuzumab HCDR2
RIYPTNGYTRYADSVKG
SEQ ID NO: 6: Trastuzumab HCDR3
WGGDGFYAMDY
SEQ ID No: 7: Trastuzumab Light chain
variable region:
DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIKRTV
SEQ ID NO: 8: Trastuzumab Heavy chain
variable region:
EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDYWGQGTLVTVSS
SEQ ID No: 9: Trastuzumab Light chain full
length:
DIQMTQSPSSLSASVGDRVTITCRASQDVNTAVAWYQQKPGKAPK
LLIYSASFLYSGVPSRFSGSRSGTDFTLTISSLQPEDFATYYCQQ
HYTTPPTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID No: 10: Trastuzumab Heavy chain full
length:
EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSREE
MTKNQVSLTCLVKGFYPSDIAVEWESNGQPENNYKTTPPVLDSDG
SFFLYSKLTVDKSRWQQGNVFSCSVMHEALHNHYTQKSLSLSPGK
SEQ ID No: 11:
LPET

1. A pharmaceutical combination, comprising the following (a) and (b):
(a) one or more conjugates having the structure of formula (i-1) and/or formula (i-2) 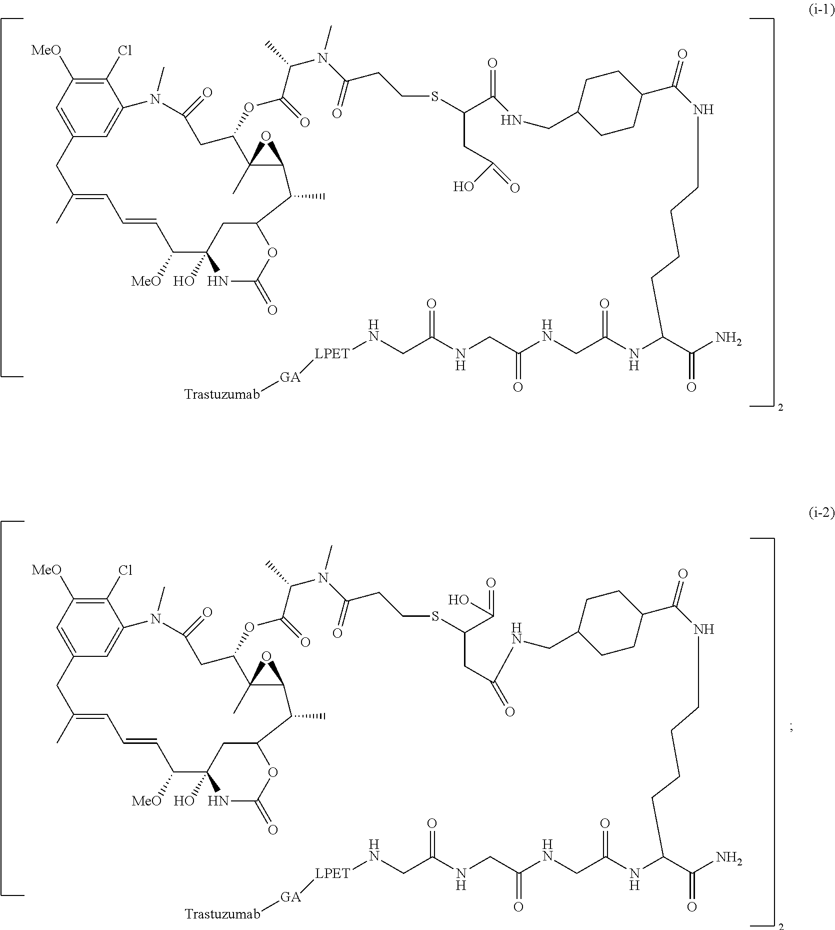
preferably, trastuzumab is connected to the rest moiety of the conjugate via the C-terminal of both of the light chains of trastuzumab;
(b) one or more additional cancer therapeutic agents selected from the group consisting of: tyrosine kinase inhibitors and chemotherapeutic agents.

2. The pharmaceutical combination of claim 1, wherein the tyrosine kinase inhibitors are selected from the group consisting of: anaplastic lymphoma kinase (ALK) inhibitors, c-Met inhibitors, HER family inhibitors, FMS-like tyrosine kinase 3 (FLT3) inhibitors, vascular endothelial growth factor (VEGF)/vascular endothelial growth factor receptor (VEGFR), fibroblast growth factor receptor (FGFR) inhibitors, platelet-derived growth factor receptor (PDGFR) inhibitors, rearranged during transfection (RET) kinase inhibitors, tropomyosin receptor kinase (TRK) inhibitors, Bcr-Abl1 inhibitors, Bruton's tyrosine kinase (BTK) inhibitors, and Janus kinase (JAK) inhibitors, optionally wherein the tyrosine kinase inhibitors are selected from the group consisting of: tucatinib, neratinib and pyrotinib, preferably tucatinib or pyrotinib.

3. (canceled)

4. The pharmaceutical combination of claim 1, wherein the chemotherapeutic agents are selected from taxanes, optionally wherein the taxanes are selected from the group consisting of: docetaxel and paclitaxel, preferably paclitaxel.

5. (canceled)

6. The pharmaceutical combination of claim 1, wherein the chemotherapeutic agents are selected from pyrimidine antimetabolites, the pyrimidine antimetabolites are selected from the group consisting of: cytarabine, capecitabine, ancitabine, gemcitabine, enocitabine, carmofur, dideoxyuridine, doxifluridine and floxuridine, preferably capecitabine.

7. The pharmaceutical combination of claim 1, wherein the one or more conjugates and the one or more additional cancer therapeutic agents are administered simultaneously, separately or sequentially.

8. A kit or a pharmaceutical composition, comprising the pharmaceutical combination of claim 1.

9. A method for preventing, alleviating or treating a cell proliferative disorder, comprising administering a subject in need of such treatment an effective amount of the pharmaceutical combination of claim 1.

10. The method of claim 9, wherein the cell proliferative disorder is a cancer.

11. The method of claim 10, wherein the cancer is a HER2-positive cancer, optionally wherein the HER2-positive cancer is selected from the group consisting of: gastric or esophagogastric cancer, breast cancer, vaginal cancer, salivary cancer, endometrial cancer, bladder cancer, non-small cell lung cancer (NSCLC), cervical cancer, ovarian cancer, colorectal cancer and pancreatic adenocarcinoma.

12. (canceled)

13. The method of claim 10, wherein the cancer is metastatic, recurrent, refractory or advanced.

14. The method of claim 9, wherein the subject in need of such treatment has received prior treatment(s);
wherein the prior treatment(s) comprises/comprise antibody therapy, chemotherapy, and/or kinase inhibitor therapy; preferably antibody therapy and/or chemotherapy,
optionally wherein the prior treatment(s) is/are selected from the group consisting of: trastuzumab, pertuzumab, tucatinib, neratinib, pyrotinib, paclitaxel, capecitabine, letrozole, and combination thereof.

15. (canceled)

16. The method of claim 14, wherein the cell proliferative disorder is a cancer; preferably, the cancer is selected from the group consisting of: gastric or esophagogastric cancer, breast cancer, vaginal cancer, salivary cancer, endometrial cancer, bladder cancer, non-small cell lung cancer (NSCLC), cervical cancer, ovarian cancer, colorectal cancer and pancreatic adenocarcinoma.

17. The method of claim 16, wherein
the cancer is breast cancer, and the subject in need of such treatment has received prior treatment(s); preferably, the prior treatment(s) is/are selected from the group consisting of: trastuzumab, pertuzumab, pyrotinib, capecitabine, palbociclib, letrozole, and combination thereof; and/or
the cancer is gastric cancer, and the subject in need of such treatment has received prior treatment(s); preferably, the prior treatment(s) is/are selected from the group consisting of: trastuzumab, pertuzumab, pyrotinib, capecitabine, palbociclib, and combination thereof.

18. The method of claim 17, wherein the subject in need of such treatment is resistant to one or more of the prior treatment(s).

19. The method of claim 9, wherein one or more conjugates and one or more additional cancer therapeutic agents are administered simultaneously, separately or sequentially.

20. The method of claim 9, wherein the conjugate is administered at an effective amount of about 5 mg/kg to 10 mg/kg per treatment cycle, and the additional cancer therapeutic agent is administered at an effective amount of about 100 mg to 600 mg once daily per treatment cycle.

21. The method of claim 20, wherein each treatment cycle of conjugate administration is independently about 1 week, about 2 weeks, about 3 weeks, about 4 weeks, about 5 weeks, about 6 weeks, about 7 weeks; and/or
wherein each treatment cycle of the additional cancer therapeutic agent administration is independently about 1 week, about 2 weeks, about 3 weeks, about 4 weeks, about 5 weeks, about 6 weeks, about 7 weeks.

22. The method of claim 20, wherein the conjugate is administered at an effective amount of about 6 mg/kg to 8.4 mg/kg per treatment cycle, and the additional cancer therapeutic agent is administered at an effective amount of about 320 mg once daily for 28 days per treatment cycle.

23. The method of claim 20, wherein the conjugate is the mixture of the conjugate of formula (i-1) and the conjugate of formula (i-2); the additional cancer therapeutic agent is pyrotinib.

24. The kit of claim 8, further comprising instructions to administer the compounds to a patient in need; wherein the conjugate is administered at an effective amount of about 5 mg/kg to 10 mg/kg per treatment cycle, and the additional cancer therapeutic agent is administered at an effective amount of about 100 mg to 600 mg once daily per treatment cycle.


SEQUENCE LISTING (11 sequences)

SEQ ID NO: 1 - protein, 11 aa, synthetic construct
  REGION 1..11: note = Trastuzumab LCDR1
RASQDVNTAVA

SEQ ID NO: 2 - protein, 7 aa, synthetic construct
  REGION 1..7: note = Trastuzumab LCDR2
SASFLYS

SEQ ID NO: 3 - protein, 9 aa, synthetic construct
  REGION 1..9: note = Trastuzumab LCDR3
QQHYTTPPT

SEQ ID NO: 4 - protein, 5 aa, synthetic construct
  REGION 1..5: note = Trastuzumab HCDR1
DTYIH

SEQ ID NO: 5 - protein, 17 aa, synthetic construct
  REGION 1..17: note = Trastuzumab HCDR2
RIYPTNGYTRYADSVKG

SEQ ID NO: 6 - protein, 11 aa, synthetic construct
  REGION 1..11: note = Trastuzumab HCDR3
WGGDGFYAMDY

SEQ ID NO: 7 - protein, 110 aa, synthetic construct
  REGION 1..110: note = Trastuzumab Light chain variable region
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTV

SEQ ID NO: 8 - protein, 120 aa, synthetic construct [sequence omitted]

SEQ ID NO: 9 - protein, 214 aa, synthetic construct
  REGION 1..214: note = Trastuzumab Light chain full length
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 450 aa, synthetic construct
  REGION 1..450: note = Heavy chain full length
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 4 aa, synthetic construct
LPET